(19) United States
(12) Patent Application Publication
(10) Pub. No.: US 2023/0348999 A1
(43) Pub. Date: Nov. 2, 2023

(54) STRATIFICATION AND PROGNOSIS OF CANCER

(71) Applicant: THE UNIVERSITY OF BRITISH COLUMBIA, Vancouver (CA)

(72) Inventors: Sohrab SHAH, Vancouver (CA); Yikan WANG, Vancouver (CA); Ali Bashashati SAGHEZCHI, Vancouver (CA); David HUNTSMAN, Vancouver (CA); Samuel APARICIO, Vancouver (CA); Andrew MCPHERSON, Vancouver (CA); Diljot GREWAL, Vancouver (CA)

(21) Appl. No.: 18/328,070

(22) Filed: Jun. 2, 2023

Related U.S. Application Data
(63) Continuation of application No. 17/751,038, filed on May 23, 2022, now abandoned.
Application No. 17/751,038 is a continuation of application No. 16/606,315, filed on Oct. 18, 2019, now abandoned.
(60) Provisional application No. 62/512,827, filed on May 31, 2017.
(60) Provisional application No. 62/488,248, filed on Apr. 21, 2017.

(51) Int. Cl. G16B 30/00; C12Q 1/6886; G16B 20/20; G16B 40/30

(52) U.S. Cl. CPC C12Q 1/6886; G16B 20/20; G16B 30/00; G16B 40/30; C12Q 2600/112; C12Q 2600/118; C12Q 2600/156; G01N 2800/52

(57) ABSTRACT

The present invention relates, in part, to methods for the stratification, prognosis, diagnosis and stratification of a cancer, such as an ovarian cancer or a breast cancer.

### CROSS-REFERENCE TO RELATED PATENT APPLICATIONS

This application is a Continuation of U.S. Application No. 17/751,038, filed on May 23, 2022, which is a Continuation of U.S. Application No. 16/606,315, filed on Oct. 18, 2019, which is a U.S. National Stage Application under 35 U.S.C. §371 of International Patent Application No. PCT/IB2018/052819, filed Apr. 23, 2018, which claims the benefit of priority under 35 U.S.C. § 119 of U.S. Pat. Application numbers 62/488,248 filed Apr. 21, 2017, and 62/512,827 filed May 31, 2017, all of which are incorporated by reference in their entireties.

### FIELD OF INVENTION

The present invention relates to the stratification and prognosis of cancer.

### BACKGROUND OF THE INVENTION

The major histotypes of ovarian cancer have distinct cellular morphologies, aetiologies, as well as molecular, genetic and clinical attributes (Kobel, M. et al., 2008; Vaughan, S. et al., 2011; Risch, H. et al., 2006; Alsop, K. et al., 2012; Berns, E. M. & Bowtell, D. D, 2012). High-grade serous (HGSC) is the most common histotype, accounting for 70% of epithelial ovarian carcinomas, and 90% of advanced stage disease and mortality. Endometriosis-associated cancers account for approximately 20% of epithelial ovarian carcinomas, including endometrioid (ENOC) and clear cell (CCOC) carcinoma histotypes (Anglesio, M. S., et al., 2011; Munksgaard, P. S. & Blaakaer, J., 2012). The major histotypes associate with distinct sets of recurrently mutated genes and aberrant mechanisms of DNA repair: for example, TP53 loss and profound genomic instability due to BRCA ½ defects are ubiquitous in HGSC (Alsop, K. et al., 2012; Ahmed, A. A. et al., 2010; Cancer Genome Atlas Research Network, 2011). CCOC and ENOC harbour ARID1A loss of function mutations (approximately 50% and 30% of cases, respectively) (Wiegand, K. C. et al.; 2010); Jones, S. et al.; 2010) variously accompanied by loss of PTEN, mutation of KRAS, CTNNB1, PIK3CA, PPP2R1A, TERT promoters (Obata, K. et al., 1998; Wu, R., et al., 2001; Campbell, I. G. et al., 2004; Kuo, K.-T. et al., 2009; Kurman, R. J. & Shih, I.-M., 2011; McConechy, M. K. et al., 2011; Nissenblatt, M., 2011; Wu, R.-C. et al., 2014) and additional properties such as microsatellite instability, mismatch repair deficiency, and hypermutation (Niskakoski, A. et al., 2013). Adult granulosa cell tumours of the ovary (GCT) are rare (4-5% of all ovarian cases), phenotypically distinct non-epithelial ovarian carcinomas, unambiguously identified through the FOXL2C134W mutation (Shah, S. P. et al., 2009).

Although these histotypes are viewed as distinct diseases, they are still usually treated with surgery and combination platinum and taxane chemotherapy (Piccart, M. J. et al., 2000; Rauh-Hain, J. & Penson, R., 2008; McAlpine, J. N. et al. 2009; Anglesio, M. S. et al. 2011; Farley, J. H., et al., 2012; Anglesio, M. S. et al., 2013). The use of PARP inhibitors in BRCA-deficient HGSC cancers represents both the first histotype-specific treatment and the first successful attempt to further stratify histotypes into meaningful treatment options (Ledermann, J. et al., 2012; Ledermann, J. et al., 2014; Mirza, M. R. et al., 2016; Swisher, E. M. et al., 2017). However, in complex disease phenotypes, gene-based biomarkers offer limited representations of underlying biology and can be complemented by more global properties.

Whole genome sequencing has shed light on intrinsic and extrinsic mutational processes via the analysis of substitution patterns and localized sequence context of mutations (Alexandrov, L. B. et al., 2013; Nik-Zainal, S. et al., 2012). Complementary insights have been gained from analysis of structural variation patterns reflective of double strand break repair mechanisms operating in various tumour types exhibiting genomic instability (Campbell, P. J. et al., 2010; Sudmant, P. H. et al., 2015), including patterns of evolution in HGSC (Ng, C. K. Y. et al., 2012). The most common structural variations include tandem duplication resulting from insertion of an adjacent identical segment, fold-back inversion forming localized inverted duplications caused by breakage fusion bridge (Campbell, P. J. et al., 2010), interstitial deletion in which the ends of multiple breaks in a chromosome are rejoined with a segment being removed, and inter-chromosomal translocations where both break-ends are on different chromosomes. The relative proportion of structural alterations attributed to tandem duplication, fold-back inversion, interstitial deletion, and other inter-chromosomal translocations provide context as a read out of specific DNA repair mechanisms operating in human cancers (Sasaki, S. et al., 2003; Yang, L. et al., 2013; Hermetz, K. E. et al., 2014).

### SUMMARY OF THE INVENTION

The present invention relates, in part, to methods for the stratification, prognosis, diagnosis, and stratification of a cancer in a subject.

In one aspect, the present invention provides a method for determining the prognosis for a cancer patient in need thereof, by: providing the genomic DNA sequence of a cancer sample from the patient; detecting structural variation patterns in the genomic DNA sequence of the cancer sample; and determining the prevalence of the structural variation patterns in the genomic DNA sequence of the cancer sample, where a high level of fold-back inversions is indicative of a poor prognosis.

The method may further include: providing the genomic DNA sequence of a normal sample; detecting structural variation patterns in the genomic DNA sequence of the normal sample; and comparing the structural variation patterns in the genomic DNA sequence of the normal sample with those in the genomic DNA sequence of the cancer sample, where the increased prevalence of fold-back inversions in the genomic DNA sequence of the cancer sample compared to the genomic DNA sequence of the normal sample is indicative of a poor prognosis.

The method may further include: detecting high-level amplifications in the genomic DNA sequence of the cancer sample, and the genomic DNA sequence of the normal sample, if present, where co-localization of the high-level amplifications and the fold-back inversions is indicative of a poor prognosis.

In another aspect, the present invention provides a method for the stratification of a cancer patient, by: providing the genomic DNA sequence of a cancer sample from the patient; detecting genomic features in the genomic DNA sequence of the cancer sample, the genomic features including single nucleotide variants, insertions/deletions, mutation signatures, and structural variants; and stratifying the patient into a cancer subgroup based on the prevalence of one or more of the genomic features.

The method may further include: providing the genomic DNA sequence of a normal sample; detecting the genomic features in the genomic DNA sequence of the normal sample; comparing the genomic features in the genomic DNA sequence of the normal sample with those in the genomic DNA sequence of the cancer sample and stratifying the patient into a cancer subgroup based on the increased prevalence of one or more of the genomic features in the genomic DNA sequence of the cancer sample compared to the genomic DNA sequence of the normal sample.

In another aspect, the present invention provides a method for diagnosing a cancer in a subject in need thereof, by: providing the genomic DNA sequence of a sample from the subject; detecting genomic features in the genomic DNA sequence of the sample, the genomic features including single nucleotide variants, insertions/deletions, mutation signatures, and structural variants, where the prevalence of one or more of the genomic features is indicative of a diagnosis of a cancer.

The method may further include: providing the genomic DNA sequence of a normal sample; detecting the genomic features in the genomic DNA sequence of the normal sample; and comparing the genomic features in the genomic DNA sequence of the normal sample with those in the genomic DNA sequence of the sample from the subject where the increased prevalence of one or more of the genomic features in the genomic DNA sequence of the sample from the subject compared to the genomic DNA sequence of the normal sample is indicative of a diagnosis of a cancer.

The methods may further include: comparing the prevalence of one or more of the genomic features to a control or reference classifier.

In some embodiments, the genomic features may include a high level of insertions and deletions or a high level of fold-back inversions.

In some embodiments, the fold-back inversions may co-localize with high-level amplifications.

In some embodiments, the methods may further include: determining a therapy for the cancer patient or the subject.

In some embodiments, a high level of fold-back inversions may stratify the cancer patient or the subject into a subgroup susceptible to a therapeutic agent targeting a DNA repair mechanism.

In some embodiments, the subgroup susceptible to a therapeutic agent targeting a DNA repair mechanism may be recalcitrant to therapy with cisplatin or a poly(ADP-ribose) polymerase inhibitor.

In some embodiments, the therapy may include sensitization to cisplatin or a poly(ADP-ribose) polymerase inhibitor, such as olaparib, niraparib, rucaparib camsylate, etc.

In some embodiments, the therapeutic agent may be a DNA polymerase theta inhibitor.

In some embodiments, the ovarian cancer patient may have been previously exposed to chemotherapy, for example, genotoxic chemotherapy.

In some embodiments, the cancer may be a breast cancer or an ovarian cancer.

In some embodiments, the ovarian cancer may be a high-grade serous carcinoma, associated with endometriosis, or a granulosa cell tumour.

In some embodiments, the ovarian cancer associated with endometriosis may be an endometrioid carcinoma or a clear cell carcinoma.

In some embodiments, the ovarian cancer may be a clear cell carcinoma subgroup susceptible to a therapeutic agent that targets an APOBEC enzyme.

In some embodiments, the ovarian cancer may be an endometrioid carcinoma subgroup susceptible to immunotherapy.

In some embodiments, the breast cancer may be a triple negative breast cancer.

In some embodiments, the cancer may be associated with a defect in a DNA repair mechanism.

In some embodiments, the DNA repair mechanism may be a homologous recombination repair mechanism.

In some embodiments, the DNA repair mechanism may be a microhomology-mediated end joining pathway.

In some embodiments, the genomic DNA sequence may be determined by whole genome sequencing.

In some embodiments, the patient or the subject may be a human.

In some embodiments, the normal sample may be a blood sample.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1 shows the integration of genomic features stratifies ovarian cancer patients, with discriminant features defining each subgroup. Heatmap showing the normalized t-score for the discriminant features in each subgroup.

FIG. 2A shows the integration of genomic features stratifies ovarian cancer patients using hierarchical clustering, where comparison between the estimated cellularities between the subgroups of each histotypes showed no significant differences. The cellularity of each sample was estimated using Titan. Student’s t-test was performed and the corresponding p-value is annotated on top of boxplots for each histotype.

FIG. 2B shows the integration of genomic features stratifies ovarian cancer patients using hierarchical clustering, where the number of clusters are determined by the ‘elbow’ rule. Plot showing the explained variance (EV; Y-axis) computed as a function of the number of clusters (X-axis) generated from hierarchical clustering. Given the threshold of EV (at 0.45 horizontal dashed line) and its increment threshold of 0.05, the optimal cluster k = 7 was identified (vertical dashed line).

FIG. 2C shows the integration of genomic features stratifies ovarian cancer patients using hierarchical clustering, with respect to the mutation load in HGSC subgroups. The mutation load for the HGSC samples in the H-HRD subgroup, on average, was higher than the H-FBI subgroup (Mann-Whitney-Wilcoxon test p-value <0.001).

FIG. 3A shows the integration/clustering of genomic features/cases in HGSC cohort only, with the importance of genomic features segregating the HGSC subgroups of H-FBI (n = 22) and H-HRD (n = 37). The genomic features (y-axis) are sorted in descending order of the average Gini score (x-axis), reflecting the importance of features in stratifying the two subgroups of HGSC tumours.

FIG. 3B shows the integration/clustering of genomic features/cases in HGSC cohort only, with the importance of genomic features segregating the HGSC subgroups of H-FBI (n = 22) and H-HRD (n = 37). Box plot showing the distribution of top six genomic features contributing to the differences between H-HRD and H-FBI. Y-axis is the value of genomic features.

FIG. 3C shows Kaplan-Meier plots showing significant differences in overall survivals between the HGSC subgroups of H-HRD and H-FBI (Log-rank test p-values = 0.0083 and 0.0108), in which samples enriched in fold-back inversions (H- FBI) had poor survival outcomes.

FIG. 3D shows Kaplan-Meier plots showing significant differences in progression free survivals between the HGSC subgroups of H-HRD and H-FBI (Log-rank test p-values = 0.0083 and 0.0108), in which samples enriched in fold-back inversions (H- FBI) had poor survival outcomes.

FIG. 4A shows the fold-back inversion profile stratifies high-grade serous ovarian cancer (HGSC) patients, demonstrating the importance of genomic features segregating H-HRD and H-FBI of HGSC tumours. Genomic features (y-axis) sorted in descending order of average Gini score (x-axis), reflecting the importance of features in stratifying subgroups.

FIG. 4B shows the fold-back inversion profile stratifies high-grade serous ovarian cancer (HGSC) patients, demonstrating the importance of genomic features segregating H-HRD and H-FBI of HGSC tumours. Box plot showing the distribution of the top six genomic features contributing to the differences between H-HRD and H-FBI. Y-axis is the value of genomic features.

FIG. 4C shows the fold-back inversion profile stratifies high-grade serous ovarian cancer (HGSC) patients, with GISTIC profiles showing the significant focal copy number amplifications for the H-HRD and H-FBI subgroups, with significantly highly amplified and deleted regions (q values <0.05) annotated.

FIG. 4D shows the fold-back inversion profile stratifies high-grade serous ovarian cancer (HGSC) patients, with GISTIC profiles showing the significant focal copy number deletions for the H-HRD and H-FBI subgroups, with significantly highly amplified and deleted regions (q values <0.05) annotated.

FIG. 4E shows Kaplan-Meier plots showing overall (left panel) and progression-free (right panel) survival between H-HRD and H-FBI of HGSC tumours. Log-rank test p-values are shown.

FIG. 4F shows Kaplan-Meier plots showing overall and progression-free survival between H-HRD (n = 16) and H-FBI (n = 24) subgroups of HGSC excluding BRCA½ germline and somatic mutation bearing cases. Log-rank test p-values are shown.

FIG. 4G shows Kaplan-Meier plots presenting overall and progression-free survival outcomes of ICGC HGSC cases in High FBI (n = 41) and Low FBI (n = 41) subgroups. Log-rank test p-values are shown.

FIG. 4H shows the distribution of BRCA mutant cases in High and Low FBI subgroups. Pearson’s Chi-squared test p-value is shown.

FIG. 4I shows distribution of the gene expression defined molecular subgroups in High and Low FBI subgroups. Pearson’s Chi-squared test p-value is shown.

FIG. 5A shows HGSC tumours stratified by fold-back inversion profile, where HGSC cases could be stratified into two subgroups based on the proportion of fold-back inversions, in which, cases with higher proportion of fold-back inversions (with reference to the median) referred to as High FBI group had statistically significant inferior overall and progression-free survival outcomes (Log-rank test p-value = 0.0187 and 0.0286) compared to the cases with low proportion of fold-back inversions (Low FBI group).

FIG. 5B shows the distribution of break distance of fold-back inversions in our HGSC cohort.

FIG. 6A shows the association between fold-back inversions (FBI) and high-level amplifications (HLAMPs) and validation on TCGA data, with the lower quantile, median and upper quantile of mean average LogR computed from FBI associated copy number (CN) amplifications in H-FBI and H-HRD subgroups at different LogR thresholds from 0.2 to 1.

FIG. 6B shows the distributions of LogR in 19q12 amplified regions in H-HRD and H-FBI subgroups. Two-sample Kolmogorov-Smirnov (KS) test p-value is shown.

FIG. 6C shows Kaplan-Meier plots for TCGA HGSC samples (n = 435) in three subgroups: (i) cases without any amplification events (No AMP); (ii) cases enriched in FBI associated amplifications (FBI-AMP High), and (iii) cases depleted of FBI associated amplifications (FBI-AMP Low). Log-rank test p value is shown.

FIG. 6D Kaplan-Meier plots for TCGA HGSC samples (n = 435) in three subgroups: (i) cases without any amplification events (No AMP); (ii) cases enriched in foldback inversion-associated amplifications (FBI-AMP High), and (iii) cases depleted of foldback inversion-associated amplifications (FBI-AMP Low). The Pvalue was calculated by log-rank test.

FIG. 6E is a bar plot showing the distribution of molecular subgroups in the No AMP, FBI-AMP High, and FBI-AMP Low subgroups. Pvalues were calculated by Pearson’s _{X}² test.

FIG. 6F is a bar plot showing the distribution of BRCA-mutant cases in the No AMP, FBI-AMP High, and FBI-AMP Low subgroups. Pvalues were calculated by Pearson’s _{X}² test.

FIG. 6G shows Kaplan-Meier plots for No AMP, FBI-AMP High and Low subgroups excluding BRCA mutant cases. Log-rank test p-value is shown.

FIG. 7 shows high-level amplification associated fold-back inversions (HLAMP-FBIs) in HGSC cell lines, with the proportion of HLAMP-FBI of the primary (TOV1369) and relapse cell line (OV1369(R2)) (dotted lines) superimposed on the distribution of HLAMP-FBI from the H-HRD and H-FBI subgroups.

FIG. 8A shows the stratification of endometriosis-associated tumours with respect to the importance of genomic features segregating C-APOBEC and C-AGE of CCOC samples. Genomic features (y-axis) sorted in descending order of average Gini score (x-axis), reflecting the importance of features in stratifying subgroups.

FIG. 8B shows the stratification of endometriosis-associated tumours with respect to the importance of genomic features segregating C-APOBEC and C-AGE of CCOC samples. Box plot showing the distribution of top six genomic features contributing to the differences between the two CCOC subgroups. Y-axis is the value of genomic features.

FIG. 8C shows the stratification of endometriosis-associated tumours with respect to the importance of genomic features segregating subgroups E-MSI and MSS of ENOC samples. Genomic features (y-axis) sorted in descending order of average Gini score (x-axis), reflecting the importance of features in stratifying subgroups.

FIG. 8D shows the stratification of endometriosis-associated tumours with respect to the importance of genomic features segregating subgroups E-MSI and MSS of ENOC samples. Box plot showing the distribution of top six genomic features contributing to the differences between E-MSI and MSS subgroups of ENOC. Y-axis is the value of genomic features.

FIG. 8E shows the stratification of endometriosis-associated tumours designated C-APOBEC.

FIG. 8F shows the stratification of endometriosis-associated tumours designated C-AGE

FIG. 8G shows the stratification of endometriosis-associated tumours designated E-MSI

FIG. 8H shows the stratification of endometriosis-associated tumours designated MSS tumours.

FIG. 9 shows a schematic tree-diagram illustrating an overview of ovarian tumour subgroupings by genomic consequences of DNA repair aberrations. GCT is characterized by a unique mutation signature identified in breast cancer (S.BC) and prevalence of FOXL2 somatic mutations. Within the ovarian carcinomas, LOH profile and homologous recombination deficiency signature (S.HRD) distinguish HGSC from non-serous histotypes. Endometriosis associated ovarian cancer histotypes are associated with ARID1A, PIK3CA and PTEN somatic mutations. Bar plots show proportions of cases harbouring mutations in a specific gene seen in a subgroup. For example, 83% of the PTEN-mutant cases are seen in ENOC while 17% are seen in CCOC. Mutation load and mismatch repair signature (S.MMR) identify three subgroups of ENOC: ultramutator, MSI and MSS. MSS subgroup is associated with high proportion of CTNNB1 and KRAS mutations. The APOBEC and age-related mutation signatures (S.APOBEC and S.AGE) stratify CCOC into two subgroups. 67% of the PPP2R1A-mutant cases are seen in the age-related CCOC group. HGSC splits into two groups: one enriched in fold-back inversions and one characterized by other types of rearrangements. Prevalence of BRCA½ germline mutations and the significance of focal copy number amplifications and losses in each HGSC subgroup are shown in bar plots. The thickness of the branch lines indicates relative sample size of subgroups in each histotype.

### DETAILED DESCRIPTION

The present invention relates, in part, to methods for the stratification, prognosis, diagnosis and stratification of a cancer, such as an ovarian cancer or a breast cancer.

Methods for determining the prognosis for a cancer patient in need thereof, may include: providing the genomic DNA sequence of a cancer sample from the patient; detecting structural variation patterns in the genomic DNA sequence of the cancer sample; and determining the prevalence of the structural variation patterns in the genomic DNA sequence of the cancer sample, where a high level of fold-back inversions is indicative of a poor prognosis.

If desired, the methods may further include providing the genomic DNA sequence of a normal sample; detecting structural variation patterns in the genomic DNA sequence of the normal sample; and comparing the structural variation patterns in the genomic DNA sequence of the normal sample with those in the genomic DNA sequence of the cancer sample, where the increased prevalence of fold-back inversions in the genomic DNA sequence of the cancer sample compared to the genomic DNA sequence of the normal sample is indicative of a poor prognosis.

If desired, the methods may further include detecting high-level amplifications in the genomic DNA sequence of the cancer sample, and the genomic DNA sequence of the normal sample, if present, where co-localization of the high-level amplifications and the fold-back inversions is indicative of a poor prognosis.

Methods for the stratification of a cancer patient may include providing the genomic DNA sequence of a cancer sample from the patient; detecting genomic features in the genomic DNA sequence of the cancer sample, the genomic features including single nucleotide variants, insertions/deletions, mutation signatures, and structural variants; and stratifying the patient into a cancer subgroup based on the prevalence of one or more of the genomic features.

If desired, the methods may further include providing the genomic DNA sequence of a normal sample; detecting the genomic features in the genomic DNA sequence of the normal sample; comparing the genomic features in the genomic DNA sequence of the normal sample with those in the genomic DNA sequence of the cancer sample and stratifying the patient into a cancer subgroup based on the increased prevalence of one or more of the genomic features in the genomic DNA sequence of the cancer sample compared to the genomic DNA sequence of the normal sample.

Methods for diagnosing a cancer in a subject in need thereof may include providing the genomic DNA sequence of a sample from the subject; detecting genomic features in the genomic DNA sequence of the sample, the genomic features including single nucleotide variants, insertions/deletions, mutation signatures, and structural variants, where the prevalence of one or more of the genomic features is indicative of a diagnosis of a cancer.

If desired, the methods may further include providing the genomic DNA sequence of a normal sample; detecting the genomic features in the genomic DNA sequence of the normal sample; and comparing the genomic features in the genomic DNA sequence of the normal sample with those in the genomic DNA sequence of the sample from the subject where the increased prevalence of one or more of the genomic features in the genomic DNA sequence of the sample from the subject compared to the genomic DNA sequence of the normal sample is indicative of a diagnosis of a cancer.

The prognostic, diagnostic or stratification information may result in the determining a suitable therapeutic regimen for the cancer patient or the subject diagnosed with a cancer, for example, a cancer associated with a defect in DNA repair; a cancer recalcitrant to therapy with cisplatin or a poly(ADP-ribose) polymerase inhibitor; or a breast or ovarian cancer. Accordingly, the methods described herein may further include administering the therapeutic regimen to the cancer patient or the subject. Suitable therapeutic regimens may include, without limitation, a therapeutic agent targeting a DNA repair mechanism or sensitization to cisplatin or a poly(ADP-ribose) polymerase inhibitor, such as olaparib, niraparib, rucaparib camsylate, etc. (against, for example, a cancer exhibiting a high level of fold-back inversions or in which fold-back inversions co-localize with high level amplifications); a therapeutic agent that targets an APOBEC enzyme (against, for example, a clear cell carcinoma); or immunotherapy (against, for example, an endometrioid carcinoma).

By a “cancer,” as used herein, is meant any unwanted growth of cells serving no physiological function. In general, a cell of a cancer has been released from its normal cell division control, i.e., a cell whose growth is not regulated by the ordinary biochemical and physical influences in the cellular environment. In most cases, a cancer cell proliferates to form a clone of cells which are either benign or malignant. Examples of cancers include, without limitation, transformed and immortalized cells, tumours, and carcinomas such as breast cell carcinomas and ovarian carcinomas. The term cancer includes cell growths that are technically benign, but which carry the risk of becoming malignant.

By “ovarian cancer,” as used herein, is meant a cancer arising from the epithelial cells of the ovary. Ovarian cancers include, without limitation, a serous ovarian cancer or high grade serous ovarian cancer, an endometriosis-associated cancer, such as an endometrioid (ENOC) carcinoma or a clear cell (CCOC) carcinoma, or an adult granulosa cell tumour of the ovary (GCT).

By a “breast cancer,” as used herein, is meant a cancer that originates in the cells of the breasts. Breast cancers include, without limitation, a triple negative breast cancer.

Different DNA lesions may invoke different, and often distinct, DNA repair mechanisms: for example, oxidative lesions, such as from reactive oxygen species, may repaired by base excision repair mechanisms; helix-distorting lesions, such as from ultraviolet radiation, may be repaired by nucleotide excision repair mechanisms; replication errors may be repaired by mismatch repair mechanisms; single strand breaks, such as from ionizing radiation and/or reactive oxygen species may be repaired by single strand break repair mechanisms, double strand breaks, such as from ionizing radiation and/or reactive oxygen species may be repaired by homologous recombination and/or non-homologous end joining mechanisms; interstrand crosslinks, such as from chemotherapy, may be repaired by DNA interstrand crosslink repair pathways, etc. In some embodiments, the DNA repair pathway may be a homologous recombination repair pathway or a microhomology-mediated end joining pathway. By a “cancer associated with a defect in DNA repair,” as used herein, is meant the malignant transformation of a cell due to a defect in a DNA repair mechanism as described herein or known in the art.

As used herein, a “subject” may be a human, non-human primate, rat, mouse, cow, horse, pig, sheep, goat, dog, cat, etc. In some embodiments, the subject may be a clinical patient, a clinical trial volunteer, an experimental animal, etc. The subject may be suspected of having or at risk for having a cancer, such as a breast cancer or an ovarian cancer, be diagnosed with a cancer, such as a breast cancer or an ovarian cancer, or be a control subject that is confirmed to not have a cancer, such as a breast cancer or an ovarian cancer. In some embodiments, the subject may have been previously exposed to chemotherapy, for example, genotoxic chemotherapy. Diagnostic methods for a cancer, such as a breast cancer or an ovarian cancer, and the clinical delineation of such diagnoses are known to those of ordinary skill in the art.

A “sample” can be any organ, tissue, cell, or cell extract isolated from a subject, such as a sample isolated from a subject having a cancer, such as a breast cancer or an ovarian cancer. For example, a sample can include, without limitation, cells or tissue (e.g., from a biopsy or autopsy) from the ovary or from an ovarian tumour, or from the breast, or any other specimen, or any extract thereof, obtained from a patient (human or animal), test subject, or experimental animal. In some embodiments, a sample may be a primary tumour sample. In some embodiments, a sample may be an untreated tumour sample. In some embodiments, a tumour sample may be a tissue biopsy sample that is fresh, frozen or formalin-fixed paraffin embedded. A sample may be from a cell or tissue known to be cancerous, suspected of being cancerous, or believed not be cancerous (e.g., normal or control). In some embodiments, it may be desirable to separate cancerous cells from non-cancerous cells in a sample. A “sample” may also be a cell or cell line, for example created under experimental conditions, that is not directly isolated from a subject.

A “control” includes a sample obtained for use in determining base-line expression or activity. Accordingly, a control sample may be obtained by a variety of ways including from non-cancerous cells or tissue e.g., from cells surrounding a tumor or cancerous cells of a subject; from subjects not having a cancer, such as a breast cancer or an ovarian cancer; from subjects not suspected of being at risk for a cancer, such as a breast cancer or an ovarian cancer; or from cells or cell lines derived from such subjects. In some embodiments, a control sample may be from the subject having a cancer, such as a breast cancer or an ovarian cancer. In some embodiments, a control sample may be from a subject other than the subject having a cancer, such as a breast cancer or an ovarian cancer. In some embodiments, a sample may be a normal blood sample. In some embodiments, a sample may be an untreated normal blood sample Accordingly, a control sample may be isolated from bone, brain, breast, colon, muscle, nerve, ovary, prostate, retina, skin, skeletal muscle, intestine, testes, heart, liver, lung, kidney, stomach, pancreas, uterus, adrenal gland, tonsil, spleen, soft tissue, peripheral blood, whole blood, red cell concentrates, platelet concentrates, leukocyte concentrates, blood cell proteins, blood plasma, platelet-rich plasma, a plasma concentrate, a precipitate from any fractionation of the plasma, a supernatant from any fractionation of the plasma, blood plasma protein fractions, purified or partially purified blood proteins or other components, serum, semen, mammalian colostrum, milk, urine, stool, saliva, placental extracts, amniotic fluid, a cryoprecipitate, a cryosupernatant, a cell lysate, mammalian cell culture or culture medium, ascitic fluid, proteins present in blood cells, etc. A control may include a previously established standard or reference classifier. Accordingly, any test or assay conducted according to the invention may be compared with the established standard and it may not be necessary to obtain a control sample for comparison each time.

By “genomic DNA,” as used herein, is meant chromosomal DNA obtained from a cell, using standard techniques known in the art or described herein. In some embodiments, genomic DNA may be from a somatic cell. In some embodiments, genomic DNA may include the whole genome or a portion thereof that is, for example, optimized for a subset of genomic regions. In alternative embodiments, genomic DNA may be the exome or a portion thereof that is, for example, optimized for a subset of genomic regions. Genomic DNA can be sequenced using standard techniques known in the art or described herein such as, without limitation, whole genome sequencing.

By “genomic features,” as used herein, is meant annotations in a genome or exome, for use in the analysis of genomic DNA. Genomic features may include, without limitation, copy number alterations (CNAs), loss of heterozygosity (LOH), single nucleotide variants (SNV), small insertions/deletions (indel), mutational signatures or profiles, structural variations (SV), etc. Genomic features can be determined as described herein or known in the art. In some embodiments, selected genomic features may be validated as described herein or known in the art, for example, by polymerase chain reaction (PCR)-based targeted amplicon sequencing.

By “structural variants,” “structural variations” or “structural variation patterns,” as used herein, is meant alterations in genomic DNA, involving segments larger than 1 kb. Structural variations include without limitation, deletions, duplications, copy number variants, insertions, inversions, translocations, frameshifts, rearrangements, etc.

By “fold-back inversion” or “fold-back inversions” is meant the breakage distance between two breakpoints in a genomic sequence. The breakage distance may be any value between about 30 bp to about 30,000 bp, for example about 100, 500, 1000, 2000, 5000, 10000, or 20000 bp. In some embodiments, two homologous short sequences, indicative of microhomology, may be present on both strands at the breakpoints of fold-back inversion. In some embodiments, the identification of fold-back inversions may be determined by standard structural variations callers, as described herein or known in the art including without limitation, deStruct (derived from nFuse; McPherson, A. et al., 2012; Gunawardana, J. et al., 2014) or Lumpy (Layer, R. M., et al., 2014) from whole genome sequence data, or Breakmer (Abo, R. P. et al. 2015) from exome sequence data. In some embodiments, the identification of an enriched or high level of fold-back inversion may be determined by the presence of fold-back inversion events in the somatic genome relative to the matched normal (germ line) genomic sequence of a subject. In some embodiments, the identification of an enriched or high level of fold-back inversion events in the somatic genome may be confirmed by comparing a subgroup of subjects to the rest of the subjects in a particular cohort. In some embodiments, the identification of an enriched or high level of fold-back inversion events in the somatic genome may be confirmed by comparing to a reference classifier. The reference classifier may be derived from a sample or collection of samples used to establish a baseline level and may include sample(s) collected from healthy person(s) or may include sample(s) collected from similar cancer patient(s) or patient subgroups.

By “frameshifting insertion/deletion,” as used herein, is meant a small genome variation that alters the reading frame of a protein coding sequence.

By a “neoantigen,” as used herein, is meant a point mutation that elicits a protein sequence that may be presented on the cell surface and recognized by the immune system.

By “microsatelite instability (MSI),” as used herein, in meant a defective mismatch repair process. Without being bound to any particular theory, the genome of a cell with MSI can accumulate variations in low-complexity regions of the genome known as microsatellites (for example, 1-6bp in length). The mutation signature associated with MSI may have a defined pattern of tri-nucleotide point mutation distribution that is detectable using the full complement of point mutations across the genome and further analysis with tools such as non-negative matrix factorization and or topic modeling as known in the art or described in, for example, Funnell et al., 2018.

By “high-level amplification” or “high-level amplifications,” as used herein, is meant amplification of segments of genomic DNA relative to a control, such as a normal genome or a reference. In some embodiments, the identification of one or more genomic amplification events may be determined as described herein or known in the art by copy number aberration callers including, without limitation, Titan, HMMcopy, etc. In some embodiments, the identification of genomic amplification events may be determined using molecular biology assays including, without limitation, probe-based DNA hybridization tools such as Affymetrix SNP Array 6.0 or array comparative genomic hybridization implementations.

In some embodiments, the co-localization of a high level amplification event with a fold-back inversion event can be determined by determining the presence of fold-back inversion breakpoints in, or near a copy number segment with LogR value above 1 to indicate the co-localization between the fold-back inversion event and the genomic amplification event. The proximity of a fold-back inversion breakpoint to a genomic amplification event to indicate co-localization may be between 0 and 50 kb kilobases apart.

In some embodiments, the tumour sample may be a cancer tumour, such as an ovarian cancer tumour or a breast cancer tumour, and the genomic amplification events may include, but not be limited to, any of the following chromosomal regions or loci; CCNE1, chr 19q21, MECOM, chr 3q26.2, PIK3CA, chr 3q26.32, CCND1, chr 11q13.3, chr 12p12.1, KRAS, chr 8q24.21, MYC, however the genomic amplification can be localized anywhere in the genome.

By “prevalence,” as used herein, is meant the occurrence of genomic features in a cancer genome relative to a control, such as a normal genome or a reference.

By “prognosis,” as used herein, is meant the likely course of a cancer, such as an ovarian cancer or a breast cancer, in a subject. In some embodiments, prognosis may include overall (OS) survival. In alternative embodiments, prognosis may include progression-free survival (PFS).

By “stratification,” as used herein, is meant the grouping of a cancer into subtypes or subgroups. In some embodiments, stratification may be based on a variety of criteria, including without limitation, molecular markers, histopathology and identification of genomic features, as described herein or known in the art.

The present invention will be further illustrated in the following examples.

### EXAMPLES

### Materials and Methods

### Patient Cohort Description

Ovarian cancer cases (n ₌ 133) were selected from the OvCaRe gynecological tissue bank (Vancouver, Canada; n ₌ 65), the CRCHUM Ovarian Cancer Tumour Bank (Montreal, Canada; n ₌ 55), and the Anatomical Pathology archives at the Jikei University School of Medicine (Tokyo, Japan; n ₌ 13). Patient consent, or waiver of consent, was approved by the respective institutional Research Ethics Boards. The BC Cancer Agency or University of British Columbia Research Ethics Board approved the overall project processes.

HGSC cases in the OvCaRe and CRCHUM Tumour Banks were selected according to the following criteria: (i) were administered platinum taxane based therapy; (ii) relapsed within 12 months (365 days) or had at least longer than 4.5 years (1642.5 days) follow-up data; (iii) had at least 50% tumour content by H&E staining and expert pathology review. All cases were re-reviewed by expert pathologists to confirm the diagnosis of HGSC. Germline BRCA1 and BRCA2 was determined for all patients through hereditary cancer screening programs. The design of cases selection as a discovery cohort was engineered to amplify biological differences by selecting cases from the extremes of the outcome distribution.

For CCOC, ENOC and GCT cohorts, OvCaRe cases were reviewed, including frozen material, by at least two expert gynecopathologists prior to inclusion in the sequencing cohort. Frozen H&E from Tokyo were also used for evaluation along with representative H&E photos and review done at the Jikei School of Medicine.

For ENOC, DAH985 and DG1288 are recurrent and both were treated with chemotherapy after their first surgery. DAH123 is an untreated sample, metastasis from a primary endometrial tumour. All HGSC, GCT, CCOC and the rest ENOC tumours are primary tumour samples.

Clinical data and follow up was provided by the Cheryl Brown Ovarian Cancer Outcomes Unit (for OvCaRe samples), or the respective providing institutions for Montreal and Tokyo cases.

### Library Construction and Sequencing

Frozen specimens with >50% tumour cellularity (based on initial slide review) were used for cryosectioning and subsequent nucleic acid extraction. Patient tumour and normal blood samples derived from primary, untreated fresh frozen tumour specimens harvested at diagnosis during standard of care debulking surgery. Germline DNA was provided from peripheral blood buffy coat on all specimens except 13 from Tokyo, where non-cancer frozen tissue was used as a germline source. DNA extraction from both matched normal (blood) and tumour samples (frozen tissue) were performed using the QlAamp Blood and Tissue DNA kit (Qiagen) and quantified using a Qbit fluorometer and reagents (high-sensitivity assay). Three lanes of Illumina HiSeq 2500 v4 chemistry for normal samples and five lanes for tumour samples were obtained. The polymerase chain reaction (PCR)-free protocol was adopted to eliminate PCR-induced bias and improve coverage across the genome.

### Sequencing Analysis

Whole genome sequencing analysis was performed to identify somatic alterations at all scales in the tumour genomes of each case, including single nucleotide variants (SNV), small insertions/deletions (indel), copy number alterations (CNA), and structural variations (SV). Revalidation through PCR-based targeted amplicon sequencing was performed for selected SNVs and SVs.

### Copy Number Alterations and Loss of Heterozygosity

Titan (Ha, G. et al. 2014; verson 1.5.5 available through R Bioconductor TitanCNA package) was performed on whole genome sequencing data to estimate cellularity, profile clonal and subclonal regions of somatic copy number alterations (CNA) and loss of heterozygosity (LOH) from the matched tumour/normal samples. All tumour samples were run with ploidy = 2 and 4 initializations. All other Titan algorithm parameters except for the following were set as default:
norm est meth = ‘map’ # estimate normal content using MAP
max iters = 50 # maximum number of EM iterations
pseudo counts = 1e-300
txn z strength = 1e6
txn exp len = 1e16
alpha high = 20000
alpah k = 15000 # prior on the copy number Gaussian variance parameter
normal params n0 = 0.5 # initial normal content
estimate ploidy = TRUE

Using the internal clustering validation measure, S Dbw validity index as a guidance, the final solution of optimal number of clusters/clones from Titan predictions was determined by manual inspection of the copy number, allelic ratio and cellular prevalence profiles from both diploid and tetraploid runs. The small CNA segments of length <5 kb were further filtered. Gene annotation for each copy number segment was performed using a python library pygenes (version 1.0.2) with human genome reference Homo sapiens GRCh37.73.gtf.

### Identification of Significantly Altered Genome Regions

GISTIC2.0 (version 2.0.21) was used to identify significantly amplified or deleted copy number aberration regions in each histotype and in each subgroup of samples. Titan-predicted copy number segments and the corresponding median LogR values were used as segmented data and the SNPs generated in Titan analysis were used as markers.

Other than the following three, other GISTIC parameters were set as default:
-conf = 0.9;
-maxseg = 2000;
-rx = 0.

The number and proportion of samples harboring deep deletion (t <-1.3), shallow deletion (-0.1 >t ≥ -1.3), neutral (0.1 ≥ t ≥ -0.1), low level gain (0.9 ≥ t >0.1), high level gain (t >0.9) were computed for every significant aberrant region (FDR q-value <0.25).

### Single Nucleotide Variant (SNV) and Indel Calling

SNVs were predicted using an updated version of mutationSeq (Ding, J. et al. 2012; version 4.3.5; model v4.1.2.npz available at http[://]compbio[dot]bccrc[dot]ca/software/mutationSeq). We also used Strelka (version1.0.13; Saunders, C. T. et al. 2012) with default parameter settings to identify somatic SNVs and indels. Both SNVs and indels were then annotated for variant effects and gene-coding status using SnpEff (Cingolani, P. et al. 2012; version 3.6b).

A set of high confidence SNVs was further identified by taking the intersection of the high probability calls predicted from mutationSeq (with probability ≥ 0.9) and the somatic SNVs predicted from Strelka. Significantly mutated genes (SMGs) were identified by MutSigCV (version 1.4; Lawrence, M. S. et al. 2013) on the entire data cohort. Genes with a false discovery rate (FDR) q <0.1 were predicted as SMGs. SNVs and indels with the following SnpEff annotations, SPLICE SITE ACCEPTOR, SPLICE SITE DONOR, NON SYNONYMOUS CODING, FRAME SHIFT, STOP GAINED, STOP LOST, in SMGs and DNA repair genes including TP53, PIK3CA, ARID1A, PTEN, PER3, KRAS, CTNNB1, FOXL2, NF1, KMT2B, PPP2R1A, PIK3R1, RPL22, POLE, RB1, BRCA1, BRCA2 were reported.

The high confidence set of SNVs were further filtered by removing the positions that fell within either of the following regions: (1) the UCSC Genome Browser blacklists (Duke and DAC), and (2) defined in the ‘CRG Alignability 36mer track’ with more than two mismatch nucleotides, requiring a 36-nucleotide fragment to be unique in the genome even after allowing for two differing nucleotides. Post processing on this set of high confidence SNVs and somatic indels from Strelka involved removing the known variants (both SNVs and indels) that were obtained from the 1000 Genomes Project (release 20130502) and dbSNP (version dbsnp 142.human 9606). The set of high confidence somatic SNVs and indels passing the above filters were then used in the downstream mutation signature analysis and feature computation.

Coding mutations were defined as positions having any of the following SnpEff annotations:

SPLICE SITE ACCEPTOR, SPLICE SITE DONOR, START LOST, NON SYNONYMOUS START, NON SYNONYMOUS CODING, FRAME SHIFT, CODON CHANGE, CODON INSERTION, CODON CHANGE PLU CODON DELETION, CODON CHANGE PLUS CODON DELETION, STOP GAINED, STOP LOST, RARE AMINO ACID.

### Mutation Signature Extraction

Trinucleotide mutation signatures were deciphered from the nucleotide substitution contexts of 133 tumour genomes using non-negative matrix factorization (NMF) with a random seeding method and the ‘brunet’ algorithm, executed by the R packages NMF (version 0.20.6) and SomaticSignatures (version 2.5.5).

NMF was run with different number of signatures (i.e., NMF rrank) from 2 to 12. For a given number of signatures, NMF was performed with 200 iterations. The goodness of fit was examined by computing the residual sum of squares (RSS) and the explained variance. The optimal number of signatures (i.e., rank = 6) was selected at which the goodness of fit converged. The inferred mutation signatures were then compared to a curated list of cancer census mutational signatures and their presence in human cancer (COSMIC: the Catalogue of Somatic Mutations in Cancer curated by the Sanger Institute, U.K, http[://]cancer[dot]sanger[dot]ac[dot]uk/cosmic/signatures; Forbes et al., 2017). The proposed aetiology of the closet match was assigned to name the inferred mutation signatures, i.e. S.APOBEC, S.POLE, S.AGE, S.BC, S.MMR and S.HRD.

To remove the random seeding bias in NMF results (i.e., to obtain stable mutation signatures), NMF with multiple random seeds was performed and a representative contribution profile for each mutation signature was computed. Briefly, with the optimal number of signatures (rank = 6) NMF was performed 2000 times and the inferred mutation signatures (basis component matrix) and their contribution profiles per sample (mixture coefficient matrix, i.e. Csignaturei, i=1:6 across 133 samples) were computed for each iteration.

Partitioning Around Medoids (PAM) method, executed by ‘pam’ under the R package cluster (version 2.0.3), was used to establish 6 clusters from the set of 2000 mixture coefficient matrices. The mean of each cluster was computed as the representative contribution of each mutation signature. The normalized contribution profiles (referred to as ‘coefficients’ in the main text), i.e. CS.AP OBEC, CS.P OLE, CS.AGE, CS.BC, CS.M M R and CS.H RD, were then used in the downstream analysis as the contribution of mutation signatures.

### Detection of Kataegis Events

Post-processed high confidence SNVs were used to identify foci of kataegis, i.e. regions of localized hypermutations, in each sample according to the criteria and method proposed in Alexandrov, L. B. et al. (2013). Briefly, for each sample, all mutations were ordered by chromosomal position and the intermutation distance (defined as the number of base pairs from each mutation to the next one) was calculated. Intermutation distances were then segmented by fitting to a piecewise constant curve based on a recursive partitioning and regression-based tree model (executed by R package rpart (version 4.1.10)) to find regions of constant intermutation distance. The minimum number of mutations that must exist in a node in order for a split to be attempted was set to six. Putative regions of kataegis were identified as those segments containing six or more consecutive mutations with an average intermutation distance of ≤ 1000 bp. The kataegic foci were further refined by retaining the regions of mutation clusters enriched for C>T and C>G mutations with a predilection for a Tp CN mutation context, i.e. %C>T|C>G >50% of total mutations at the kataegic foci, of which %TpCN context >50%.

### Structural Variation Prediction

Rearrangement breakpoints were predicted using lumpy (version 0.2.13; Layer, R. M., et al., 2014) executed by SpeedSeq version 0.1.0 (Chiang, C. et al. 2015) and destruct (version 0.4.5) derived from nFuse (McPherson, A. et al., 2012; Gunawardana, J. et al., 2014), available at https[://]bitbucket[dot]org/dranew/destruct. In brief, destruct extracted discordant and non-mapping reads from BAM files and realigned the reads using a seed and extend strategy. Split alignment across a putative breakpoint was attempted for reads that did not fully align to a single locus. Discordant alignments were clustered according to the likelihood they were produced from the same breakpoint. Multiple mapped reads were assigned to a single mapping location using previously described methods (McPherson, A. et al., 2011; Hormozdiari, F. et al. 2010). Finally, heuristic filters removed predicted breakpoints with poor discordant read coverage of sequence flanking predicted breakpoints.

A stringent 3-step filtering criterion to identify high confidence breakpoint calls for downstream analysis was applied as follows:
Step 1: breakpoints that were predicted by both algorithms, lumpy and destruct, were taken.
Step 2: we removed (1) the breakpoints from the poor mappability regions, (2) events with break distance ≤ 30 bp, (3) breakpoints annotated as deletion with breakpoints size <1000. Furthermore, only high confidence breakpoints that had at least five supporting reads in tumour and no read support in the matched normal sample were used in the analysis. The breakpoints were further filtered by removing the positions in either of the following regions: (1) UCSC Genome Browser blacklists (Duke and DAC), and (2) defined in the ‘CRG Alignability 36mer track’ with more than two mismatch nucleotides, requiring a 36-nucleotide fragment to be unique in the genome even after allowing for two differing nucleotides.
Step 3: predictions with small break distance and low number of support reads in tumour samples were excluded. We designed a targeted deep sequencing PCR experiment to inform the filtering criteria for this step.

### Rearrangement Classification

Breakpoints were classified by the orientation type and rearrangement type. Orientation type refers to the relative position and orientation of the break-ends in the genome and consists of 4 categories: deletion, duplication, inversion and translocation. Translocation breakpoints are those for which the break-ends are on different chromosomes, deletion breakpoints are those resulting from removing a segment of a chromosome and rejoining the free ends, duplication breakpoints are those resulting from a copy of a segment being inserted before or after the segment (tandem duplication), inversion breakpoints refer to one of the two breakpoints resulting from excision, inversion and reinsertion of a segment.

Rearrangement type refers to the type of rearrangement event that produced the breakpoint, where a rearrangement can be the result of one or more breakpoints. Rearrangement type consists of 6 categories: balanced, deletion, fold-back, inversion, duplication and unbalanced. Balanced rearrangements are any set of breakpoints that preserve the number of copies of adjacent chromosomal segments. We identify balanced rearrangements as alternating cycles in the breakpoint graph as described in McPherson, A. et al., 2011. Included in balanced rearrangements are reciprocal translocations, balanced insertions, and inversions greater than 1 Mb in size for which both breakpoints have been identified. Inversions less than 1 Mb in size are given the rearrangement type of inversion. Deletion and duplication rearrangement types are single breakpoint events, maximum 1 Mb in size, for which those breakpoints have not been identified as part of a balanced rearrangement. Fold- back rearrangements are inversion type breakpoints, maximum 30 kb in size, that have not been identified as part of an inversion or other balanced rearrangement. These breakpoints are termed fold-back as they imply an operation, duplication of a chromosome arm and subsequent joining of the two arms with opposing orientation, that results in the DNA sequence folding back on itself. The remaining set of unclassified breakpoints are given the rearrangement type of unbalanced.

### Genomic Feature Computation for Clustering

We generated 20 genomic features for integrative clustering ovarian cancer tumours based on copy number aberrations (CNAs), mutation profiles and structural variation characteristics, as shown in Table 1), including 6 mutation signatures: S.APOBEC, S.POLE, S.AGE, S.BC, S.MMR, and S.HRD; 6 rearrangement types and 1 homology length: Fold-back Inversion, Inversion, Tandem Duplication, Deletion, Rearrangement, Balanced Rearrangement, Unbalanced Rearrangement, and Homology>=5 bp; 3 copy number aberrations: CN.Amplification, CN.Loss, and CN.LOH; and 4 mutation variant types: Nonsynonymous, Splice site, Stop.Lost/Gained, and Frameshift.

TABLE 1

Descriptions of genomic features.

| | |
|---|---|
| Features | |
| S.APOBEC | Contribution of a mutation signature, aka, COSMIC Signature.13 - APOBEC |
| S.POLE | Contribution of a mutation signature, aka. COSMIC Signature.10 - POLE |
| S.AGE | Contribution of a mutation signature, aka. COMIC Signature.1 - AGE |
| S.BC | Contribution of a mutation signature, aka. COMSIC Signature.8 |
| S.MMR | Contribution of a mutation signature, aka. COSMIC Siganture.6 - MMR |
| S.MRO | Contribution of mutation signature, aka. COSMIC Signalure.3 - HRD |
| Foldback.lnversion | Proportion of foldback inversions |
| Inversion | Proportion of inversions |
| Tandem.Duplication | Proportion of tandem duplications |
| Deletion.Rearrangement | Proportion of deletions |
| Balanced.Rearrangement | Proportion of balanced rearrangements |
| Unbalanced.Rearrangment | Proportion of unbalanced rearrangements |
| Homology>∝5bp | Proportion of rearrangements with microhomology of >∝ 5 bp. |
| CN.Amplification | Proportion of genome showing copy number high-level amplification (copy number > ploidy+2) |
| CN.Loss | Proportion of genome showing copy number loss (homozygous deletion or deletion LOH) |
| CN.LOH | Proportion of genome harbouring dominant LOH events |
| Nonsynonymous | Proportion of non-synonymous coding mutations |
| splicesite | Proportion of splice site mutations |
| Stop.Lost/Gained | Proportion of stop lost or stop gained mutations |
| Frameshift | Proportion of frameshifting indels |

Three CNA-related features included the proportion of genome harboring loss of heterozygosity (LOH), the proportion of genome harboring copy number high-level amplification (CN.Amplification) and the proportion of genome harboring copy number loss (CN.Loss). For each sample, LOH was computed as the total length of copy number segments inferred by Titan with Titan calls in dominant clonal DLOH, NLOH or ALOH divided by total length of the genome. CN.Amplification was computed as the total length of copy number segments in which the estimated total copy number > estimated ploidy (to the nearest one) + 2 divided by the total length of the genome. CN.Loss was computed as the total length of copy number segments associated with Titan calls in DLOH or HOMD divided by the total length of the genome.

The mutation profiles comprised of the contribution of six mutation signatures and the proportion of four types of mutations: non-synonymous coding mutations, stop-gained/loss mutations, splice-site mutations and frameshifts. The contribution of mutation signatures was the normalized representative contribution of each mutation signature. For each sample, the proportions of non-synonymous coding, stop-gained/loss, splice-site, and frameshift mutations with SnpEff effect in the following categories were computed: NON SYNONYMOUS CODING for Nonsynonymous; SPLICE SITE ACCEPTOR or SPLICE SITE DONOR for Splice-site; STOP GAINED or STOP LOST for Stop.Lost/Gained; and FRAME SHIFT for Frameshift.

The structural variation characteristics were defined by the types of rearrangements and length of homology associated with each rearrangement. The proportion of six rearrangement events defined as Fold-back (Foldback.lnversion), Duplication (TandemDuplication), Deletion (DeletionRearrangement), Balanced (BalancedRearrangement), Unbalanced (UnbalancedRearrangement), and Inversion was computed for each sample. For the cases with no breakpoints passing the stringent criteria (as described herein), the proportion of rearrangement events was treated as NA. The proportion of rearrangements in each sample associated with large homology, i.e. homology size ≥ 5 bp (Homology>_ 5 bp), was computed.

The 20 genomic features for 133 tumour samples were combined to generate a feature matrix, representing genomic characteristics of the patients. The missing values were imputed in the feature matrix, i.e. proportion of rearrangement events, using impute.knn function from the R package impute (version 1.44.0) with default parameter settings. Each feature in the matrix was then scaled by subtracting the values from its mean and then dividing the values by its standard deviation.

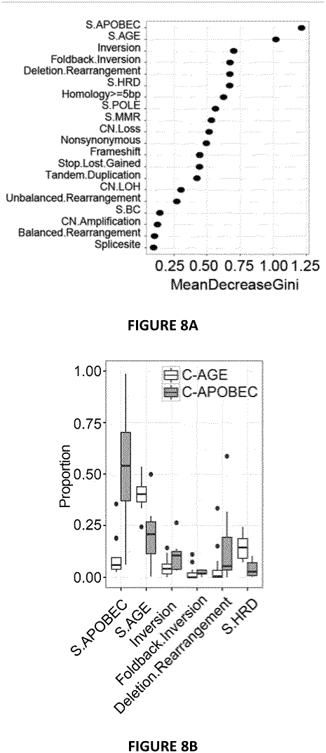

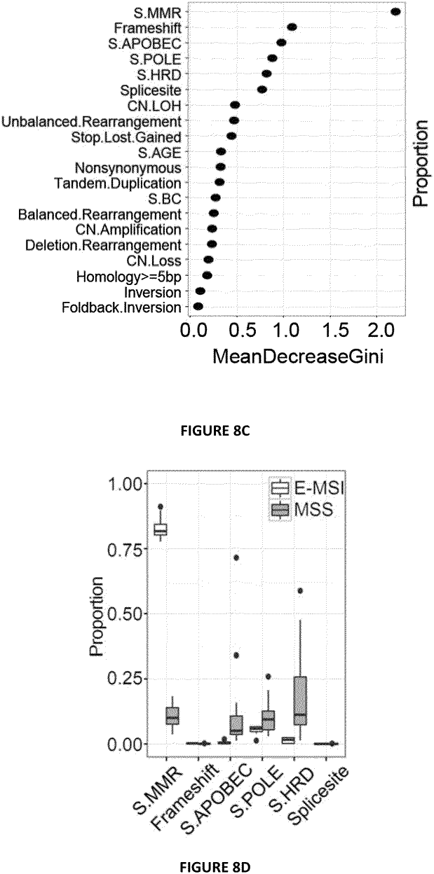

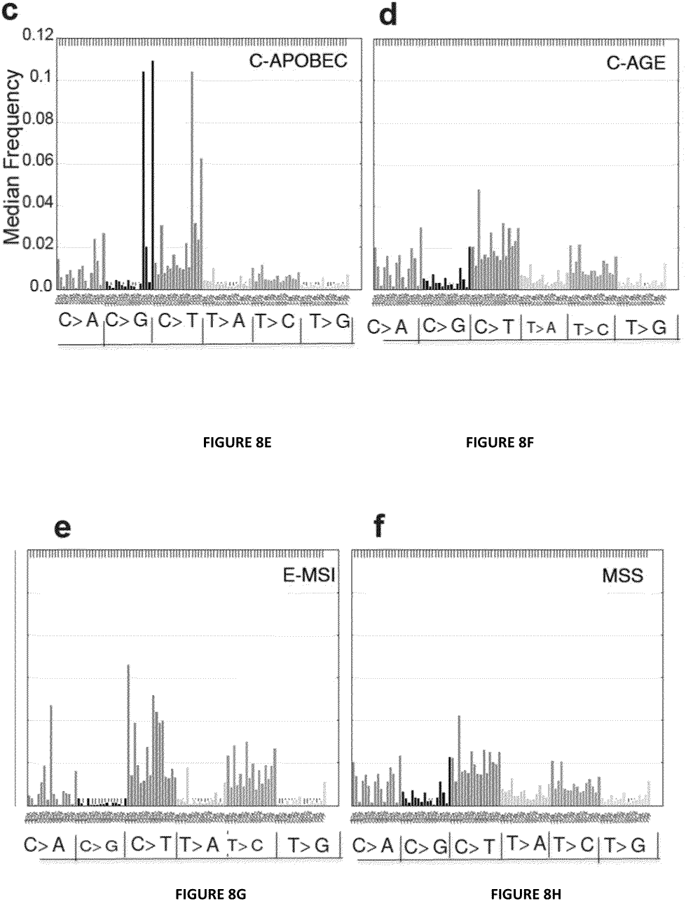

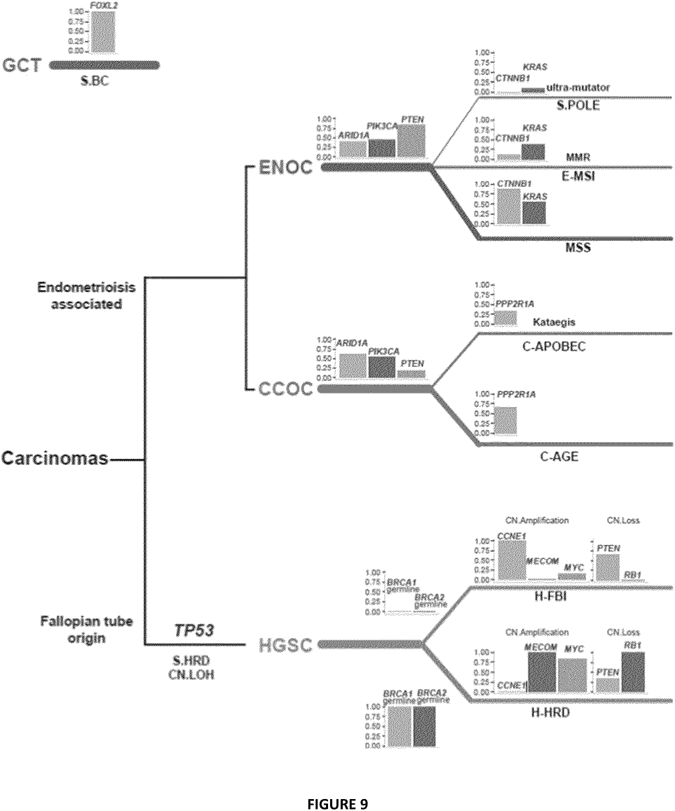

Hierarchical clustering analysis (using R package pheatmap (version 1.0.8)), using ‘manhattan’ distance measure and ‘ward.D’ agglomeration method, was performed on the feature matrix to determine the subgroupings of 133 patients. The cut-off selected for the dendrogram was determined by assessing the percentage of explained variance (EV) and its increment for a given number of cluster k using the ‘elbow’ rule. Given the distance matrix and the hierarchical clustering, the css.hclust function (R package GMD (version 0.3.3)) was used to compute the sum-of-squares. The percentage of variance explained was computed as the ratio of total between-group variance to the total sum of squares of the data (data not shown). Following the ‘elbow’ rule, the elbow. batch function was used for clustering evaluation and the optimal number of clusters (i.e., seven clusters) was selected with threshold of the EV ₌ 0.45 and the threshold of the increment in EV ₌ 0.05 (FIG. 8E).

### Fold-back Inversion Associated With HLAMP

Given breakpoints of rearrangement (from destruct and lumpy) and copy number aberrations (from Titan) identified from WGS data, we computed the average LogR values of copy number gains for SNPs within a 100 kb window of a breakpoint (i.e. 50 kb on each side of a breakpoint). Afterwards, the mean value of the average LogR corresponding to each type of rearrangement for a given case was computed. The lower quantile, median and upper quantile was then calculated, separately, for cases in H-HRD and H-FBI.

### Fold-Back (as a Single Feature) Stratified HGSC Cases

### Discovery HGSC Cases

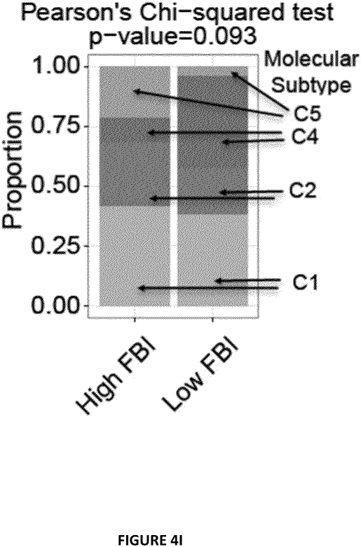

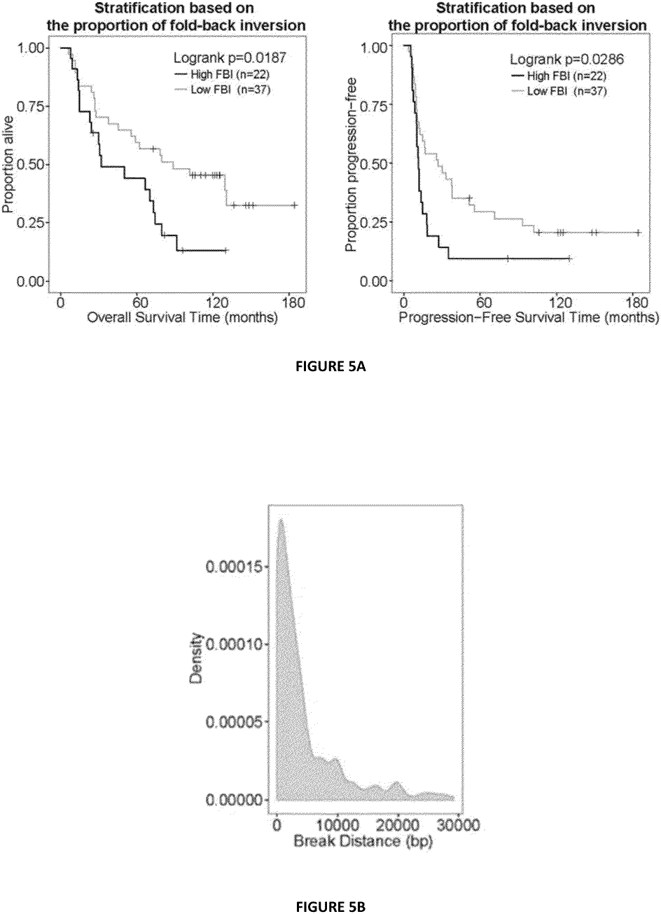

The cases were stratified into two groups based on the median value of the fold-back inversion proportion: cases with proportion of fold-back inversions > its median value were in the group of High FBI and the rest cases were in the group of Low FBI. Log-rank test was performed on the two groups to determine the significance in the difference between their survival outcomes (FIG. 5A).

### ICGC HGSC Cases

ICGC HGSC cohort structural variants and clinical outcome data (release 17) were downloaded from ICGC data portal. Only primary tumour samples were included. Inversions with breakage distance ≤ 30000 bp were reclassified as fold-back inversions. The proportion of fold-back inversions was computed for each sample. The ICGC HGSC cases were then stratified into two groups based on the median value of the fold-back inversion proportion: cases with proportion of fold-back inversions > its median value were in the group of High FBI and the rest of cases were in the group of Low FBI. Gene expression molecular subtypes and BRCA status for the ICGC HGSC cases were available from Patch, A.-M. et al. (2015). Log-rank test was performed on the two groups to determine the significance in the difference between their survival outcomes. In addition, using the same procedures that we analyzed our in-house HGSC cohort, we profiled the copy number aberrations and rearrangement events from the raw BAM files for 62 (out of 82) ICGC HGSC cases for which both matched tumour/normal BAM files were available through the ICGC Data Portal https[://]dcc[dot]icgc[dot].org.

### Prediction of Structural Variations From TCGA Ovarian Exome Sequencing Data

TCGA high-grade serous ovarian cancer cases were analyzed to determine whether the co- occurrence of amplifications (AMPs) and fold-back inversions stratify cases into subgroups with distinct survival outcomes using the following criteria:
A set of n = 435 TCGA ovarian serous cystadenocarcinoma cases with complete data of hg19 exome BAM files, copy number, and clinical data was selected for this analysis. The copy number SNP array data and clinical data for these cases were downloaded from the TCGA Pancancer project under Synapse (https://www.synapse.org/) with Synapse ID: syn1461171. The corresponding exome BAM files were downloaded from the British Columbia Cancer Agency’s Genome Sciences Centre (GSC) servers, which host the TCGA sequencing data.
Genes associated with copy number LogR ≥ 1 were extracted for each case. The genomic positions for the genes were obtained from UCSC. A total of 360 cases were found to harbour amplifications in at least one gene (in other words, 75 cases were found to have no AMP events).
To identify structural variations in copy number amplified regions, BreaKmer (version v0.0.6; Abo, R. P. et al., 2015), with default parameter settings, was performed on the 360 cases.
Post-processing on the BreaKmer predicted rearrangements included (i) removing undefined structural variation subtype, i.e. SV subtype = “None”; (ii) keeping the rearrangement events supported by at least 6 discordant reads counts; (iii) genomic breakpoints associated with each rearrangement with at least 60 read depth and 2 split reads were included in downstream analysis; and (iv) inversions with break distance ≤ 30000 bp were further classified as fold-back inversions.
For each case, we identified all the AMP regions harboring fold-back inversions and then computed the average LogR of these regions for each case.
Taking the median of the average LogR as the boundary, the 360 cases were split into two subgroups: cases with average LogR > median average LogR (FBI-AMP High, n = 174) and cases with average LogR ≤ median average LogR (FBI-AMP Low, n = 186).
By incorporating the set of cases with no AMP (n = 75), a survival analysis was performed on the three subgroups using R package survival (version 2.38.3). The Kaplan-Meier estimator and the log-rank test were computed to compare the survival outcomes between the three subgroups.

### HGSC Cell Line Sequencing and Analysis

Two HGSC cell lines derived from either solid tumour tissue (TOV) or ascites (OV) of the same patient 1369 in Le′tourneau, I. J. et al. (2012), were selected for whole-genome sequencing. The cell line TOV1369 was derived from the primary tumour sample, collected at diagnosis and OV1369(R2) was derived from the relapse sample that had been treated with chemotherapy. The corresponding IC50 values for carbopolatin and olaparib were reported and the methods were as described and used in Fleury, H. et al. (2016) and Fleury, H. et al. (2015). The cell lines did not have corresponding matched normals. However, similar to matched tumour/normal analysis, both destruct and lumpy with some modifications were used to predict breakpoints. We ran destruct on a pool of samples including the cell line samples and eight normal samples (DAH290, DG1316, DG1230, DG1023, DAH145, DAH123, DG1331 and DAH168) chosen at random, with two samples from each of the 4 ovarian cancer types under consideration. The destruct run resulted in a set of breakpoint predictions for the pool of datasets, and, for each prediction, the number of reads supporting that prediction in each dataset. Predictions supported by at least one read in any of the normal samples were marked as germline/artifact and filtered. Additionally, predictions supported by at least one read in two or more distinct cell line samples were marked as artifacts and filtered. Further filtering was identical to the matched normal destruct analysis. In addition, lumpy (single-sample mode) was performed on the cell line samples. Highly filtered breakpoints that were predicted by both destruct and lumpy, were used for computing the profile of rearrangement events.

In addition, copy number aberrations were profiled from the WGS data of the cell lines using HMMCopy (Ha, G. et al., 2012). High-level amplification associated with fold-back inversion (HLAMP-FBI) were identified and the corresponding proportion of the HLAMP-FBI events was then computed.

### PCR Validation of Breakpoints Prediction

### Target Selection

An experiment was designed to comprehensively validate the presence of fold-back inversions and other rearrangement breakpoints in a single sample. Sample DAH208 was used because it harbored a wide spectrum of rearrangement predictions including a high number of fold-back inversions at low prevalence (i.e., low read counts supporting the prediction) and some high prevalence events (i.e., high read counts supporting the prediction). The primary aims of the experiment were to determine if the many fold-back inversions supported by low read counts were true rearrangements, false positive artifacts, or very low prevalence sample-specific events and to identify features of fold-back inversions and other rearrangement breakpoints that could discern true from false positives. The following 9 categories of breakpoints were targeted, with the specified number of events for each category:
5 deletions breakpoints
5 duplications breakpoints
21 general fold-backs breakpoints
10 high break distance breakpoints
10 high homology breakpoints
15 high num reads breakpoints
10 low break distance breakpoints
10 low homology breakpoints
10 unbalanced breakpoints

Categories were defined as follows: High read count fold-backs had at least 5 WGS reads. Low break distance fold-backs were fold-backs with breakends within 4 nucleotides, and high break distance greater than 100. High and low homology was defined as greater than 10 and less than or equal to 3, respectively.

### Bioinformatics of Validation Approach and Analysis Results

Targeted deep-sequencing was performed according to internal lab standard operating procedures as described in Eirew, P. et al. (2014) and McPherson, A. et al. (2016), and the respective manufacturers’ specifications. PCR and MiSeq sequencing produced 151X151 bp paired end reads, 3953239 for the normal sample and 12790459 for the tumour sample. Reads were aligned to predicted breakpoint sequences using bwa version 0.7.12. Paired end reads were discarded unless at least 100bp of each read aligned to the same breakpoint sequence, within 5bp of the expected start location given the location of the primers. Passing read alignments were counted for each breakpoint. Read counts were less than 98 for the normal sample. The read count distribution for the tumour sample was multi-modal, with read counts less than 100 for some breakpoints and greater than 1000 for others. Based on the distribution of read counts in the normal sample we selected a presence/absence threshold of 100 reads for both tumour and normal samples. For predictions that passed the presence/absence threshold of 100, tumour read counts were greater than 2282, with median 84364 and 1st and 3rd quartiles at 23320 and 112015 respectively. We successfully validated 5/5 deletions, ⅘ duplications, 2/10 unbalanced and 8/15 high read count fold-backs (data not shown). None of the predictions with less than 100 breakpoint distance validated. Homology of successfully validated events was 6 or less, and higher homology events did not validate. Based on this, breakpoint prediction filtering criteria was adjusted to include breakpoints with read support ≥ 5 and break distance ≥ 30. This resulted in true positive rate of 90.5% (19 true SVs out of 21 predictions).

### Validation of Single Nucleotide Variants (SNVs)

### Targeted Library Construction and Sequencing

To establish the sensitivity and specificity of the SNV prediction pipeline, validation experiments were performed on all 59 HGSC tumor/normal pairs. 192 predicted somatic SNVs were selected per case as candidates for deep sequencing, which included all the somatic coding SNVs and, if the number of coding SNVs was less than 192, randomly selected high confidence non-coding SNVs to reach to 192 targets per case.

Whole genome amplification (WGA) was performed on matched tumour/normal samples. 192 case-specific primers were designed with an average primer length of 40 bases, optimization and amplicon generation. Primer quality control (QC) and forward and reverse PCR amplification was performed. Genomic libraries were created for Illumina sequencing using the plate-based small gap library construction. Libraries were indexed, pooled and sequenced on an Illumina HiSeq using 250 base PET lanes to a median depth >5000x. 42 of the initial 59 libraries passed WGA, QC and PCR amplification and were carried forward for downstream targeted deep sequencing analysis.

### Targeted Deep Sequencing Analysis and Results

The FASTQ files containing the sequenced amplicon reads were aligned to the human reference genome GRCh37-lite using bowtie2 v2.0.2 (Langmead, B. & Salzberg, S. L., 2012). The minimum base quality was set to 10, and the minimum mapping quality to 20. For each position in an amplicon region, the number of reads corresponding to the predicated variant allele and the reference allele were extracted. GATK v3.1-1 (McKenna, A. et al., 2010; DePristo, M. A. et al., 2011; Auwera, G. A. et al., 2013) was used to call variants within each amplicon. The sequencing error rate for each case was computed as the average variant allele frequency for within-amplicon positions in tumour/normal samples. A low-coverage threshold of 50 reads was applied, and the Binomial exact test utilized to infer the presence/absence of the target, as described in Shah, S. P. et al. (2012) and Shah, S. et al. (2009). P-values were adjusted using the Benjamini-Hochberg procedure, with a false discovery rate of 0.001. Following this, variant calls in the patient tumor samples were compared to the matched normal and each position was assigned a mutation status as follows:
no coverage, if either the normal or tumor samples had no coverage
low coverage, if either the normal or tumor sample had low coverage, i.e., coverage ≤ 50
wildtype, if both the normal and tumor samples did not have a variant (called ‘absent’)
somatic, if a variant was absent in the normal sample and was present in the tumor with an allelic frequency >0.05; or if the germline was present with an allelic frequency <0.05 and the variant in the tumor was present with a high allelic frequency
probable somatic, if normal had low coverage but with 0 allelic frequency while the variant in the tumor was present with high allelic frequency >0.05
germline, if the germline was present and the germline allelic frequency was >0.05
unknown otherwise

The positions annotated as ‘somatic’ or ‘probable somatic’ were considered as being validated. Out of the 42 cases passing QC and PCR, validation rate was computed as (the number of validated somatic SNVs) divided by (the total number of SNVs for which there was coverage to determine the mutation status). Overall, the average validation rate was 94% per case.

### Nanostring Molecular Subtypes

Total RNA was extracted from tumour specimen using standard protocols. For fresh frozen, RNA tissue was cryo-sectionned and then processed using the Qiazol-column method from the Qiagen miRNeasy kit according to manufacturers’ recommendations (Qiagen). For FFPE derived tissue, 3x 10 um scrolls of FFPE tissue were cut, deparaffinized using Xylene and processed following the recommendation of the Qiagen miRNeasy FFPE kit (Qiagen) with an extended (45 min) proteinase K digest at 55C. All RNA was quantified on a NanoDrop spectrophotometer and considered of sufficient quality for analysis if the Absorbance (260/280 nm) was between 1.7-2.1.

NanoString gene expression was conducted according to manufacturer’s recommendations using 500 ng total RNA for FFPE derived specimens and 100 ng total RNA for fresh/frozen derived specimens. 365 genes were selected from a cross-reference of literature derived biomarkers (Kalloger, S. E. et al., 2011; Anglesio, M. S., et al., 2011a; Madore, J. et al. 2010; Ko¨bel, M. et al., 2009a; Ko¨bel, M. et al. 2009b; Ko¨bel, M. et al. 2008) and differentially expressed genes, between molecular subtypes and/or histological subtypes using publicly available expression datasets (Helland, A°. et al., 2011; Anglesio, M. S. et al., 2011b; Anglesio, M. S. et al., 2008; Tothill, R. W. et al., 2008; Cancer Genome Atlas Research Network, 2011; Ramakrishna, M. et al., 2010; Hendrix, N. D. et al., 2006). Data were normalized with nSOLVER software (NanoString Technologies) using the geometric mean of ACTB, SDHA, PGK1, RPL19, and POLR1B counts. To allow for multiple clustering methods resulting in similar molecular subtypes to those reported previously, weighted consensus of clustering methods was used including NMF and Kmeans methods discussed in original defining studies of ovarian HGSC molecular subtypes (Tothill, R. W. et al., 2008; Cancer Genome Atlas Research Network, 2011). Herein, each clustering method was run on the log base 2 normalized counts, iteratively 1000x to establish 4 groups (k ₌ 4). We then merged the consensus class assignment of each clustering method to establish each final class and used marker genes from TCGA37 and Tothill, R. W. et al., 2008) studies to assign class names or the equivalents. The concordance of this method was verified to the original Tothill and TCGA studies by reducing each of the datasets to the same genes selected for NanoString and then applying the consensus method. Concordance was 81 % (Tothill, R. W. et al., 2008) and 76% (TCGA), with the resulting overall survival comparison being highly similar to the original reports.

### Prediction of Neoantigens in ENOC

### HLA Predictions

To predict human leukocyte antigen (HLA) genotyping, OptiType (Szolek, A. et al., 2014) was performed with default parameters on both tumour and normal aligned WGS BAM files for each ENOC case. The two highest-scoring four-digit predictions for the HLA-A locus were retained. Predictions were consistent between all tumour/normal pairs.

### MHC-Ibinding Prediction

A modified version of the pVAC-Seq (Hundal, J. et al., 2016) pipeline was used for MHC-I binding prediction. A list of 7864 processed nonsynonymous somatic SNVs along with the corresponding wildtype and variant peptide sequences was used as input for netMHC 3.4 (Nielsen, M. et al., 2003; Lundegaard, C. et al., 2008; Lundegaard, C., Lund, O. & Nielsen, M., 2008) and netMHCpan 2.8 (Hoof, I., et al., 2008; Nielsen, M. et al., 2007). Given the HLA allele predictions described above, eight to 11-mer peptides were predicted using default settings. When available for a given HLA allele, the predictions from netMHC were used; otherwise, those from netMHCpan were used. Peptides with an IC50 value <500 nM and better affinity than the corresponding wild-type peptide were kept for further analysis.

### Filtering

The further filtering was performed on the predicted epitopes according to the criteria described in Hundal, J. et al., 2016. The predicted epitopes remained in the downstream analysis for corresponding to variants with normal coverage ≥ 5X, normal variant allele fraction (VAF) ≤ 2%, tumour DNA and RNA coverage ≥ 10X, tumour DNA and RNA VAF ≥ 10%, and gene FPKM >1. VAF values were determined from WGS BAM files with MutationSeq, and from RNA-seq BAM files with ASEReadCounter (Castel, S. E., et al., 2015). Cufflinks (version v2.1.1; Trapnell, C. et al., 2010; Roberts, A., et al., 2011a; Roberts, A., et al., 2011b; Trapnell, C. et al., 2013) was performed on each case to compute Gene FPKM values.

### General Statistical Analysis

We applied shrinkage discriminant analysis to determine the feature ranking for each subgroup using the function sda.ranking with default setting in the R package sda v1.3.7. The over- representation of each histotype per subgroup was tested using one-sided Fisher’s exact test, performed by the R function fisher.test, with the fisher exact p-value adjusted using Benjamini- Hochberg method. Feature importance measure was computed for the subgroups of histotypes using the pRF function from the R package pRF version 1.2 with ntree=50 and n.perms=100, which estimates the statistical significance of the Decrease in Gini Coefficient metrics of random forest feature importance. The significance of differences between features of different subgroups were tested using Student’s t-test (two-tailed, confidence level = 0.95) with the p- values adjusted by Benjamini-Hochberg correction. The enrichment of molecular subtypes and BRCA status between the two HGSC subgroups were tested using Chi-squared test, performed by R function chisq.test. The differences in the distribution of the average LogR values per type of structural variants between the two HGSC subgroups were tested using Mann-Whitney-Wilcoxon test (two-tailed) performed by wilcox.test function, from the R package stats (version 3.2.3). The Kaplan-Meier estimator and the log-rank test were computed, using R package survival (version 2.38.3), to compare the survival outcomes between HGSC subgroups. The difference in the number of immunogenic epitopes generated in the ENOC MSI cases and MSS cases was tested using Kruskal-Wallis test, performed by R function kruskal.test.

### EXAMPLES

### Example 1 - Patterns of Somatically Acquired Genomic Variants in GCT, CCOC, ENOC and HGSC Ovarian Cancers

One hundred and thirty-three ovarian cancer patients with histologically confirmed HGSC (n = 59), CCOC (n = 35), ENOC (n = 29), and GCT (n = 10) were included in this study. Clinical follow up data (including overall and progression-free survivals) for HGSC, ENOC and CCOC cases were also recorded. BRCA1 methylation and BRCA½ germline status were determined for all HGSC patients through hereditary cancer screening programs. Microsatellite instability (MSI) testing performed on all tumour DNA using five repeated loci confirmed MSI in 28% of ENOC cases (n ₌ 8) and was low or negative in all other cases.

Tumour and matched normal DNA samples from each patient were subjected to whole genome sequencing with median coverage of 51x and 37x for the tumour and matched normal, respectively. Somatic alterations at all scales were identified in the tumour genomes of each case, including single nucleotide variants (SNVs), small insertions/deletions (indels), copy number alter- ations (CNAs), and structural variations (SVs) (revalidation through PCR-based targeted amplicon sequencing was performed for selected SNVs and SVs). Wide variation both within and between histotypes was observed for all event types. For example, the number of SNV events distributed as follows: (GCTs 568-2471 per tumour, median 1433; CCOCs 481-18268 per tumour, median 2693; ENOCs 1165-596135 per tumour, median 3928; HGSCs 1595-16058 per tumour, median 4812). Given this wide variation, we consequently investigated if global patterns of somatic genomic variation could be exploited to stratify cases.

### Example 2 - Genomic-Based Stratification of Ovarian Cancer Histotypes

Twenty genomic features were computed for each case from somatic SNVs, indels, CNAs and SVs including six previously described mutation signatures (Alexandrov, L. B. et al. 2013), four additional SNV/indel properties, seven SV features, and three CNA properties (detailed descriptions of the 20 features in Table 1). The consensus coefficients were determined (C_{S}._{BC}, C_{S}._{M} _{M} _{R}, etc.) for the six signatures (described herein) required to explain the SNV mutational repertoire in each sample). CNA features were inferred as the proportion of the genome affected by amplifications, deletions and loss of heterozygosity (LOH). SV features were determined by the relative proportion of balanced rearrangements, deletion rearrangements, tandem duplication, fold-back inversion, inversion, and unbalanced rearrangements in all SVs for each case.

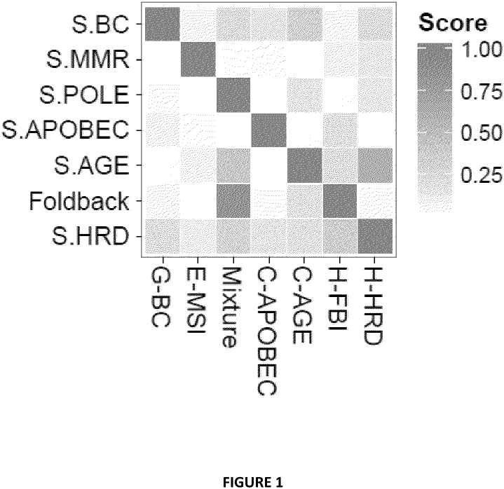

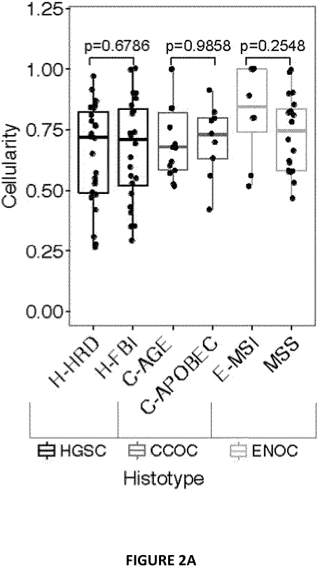

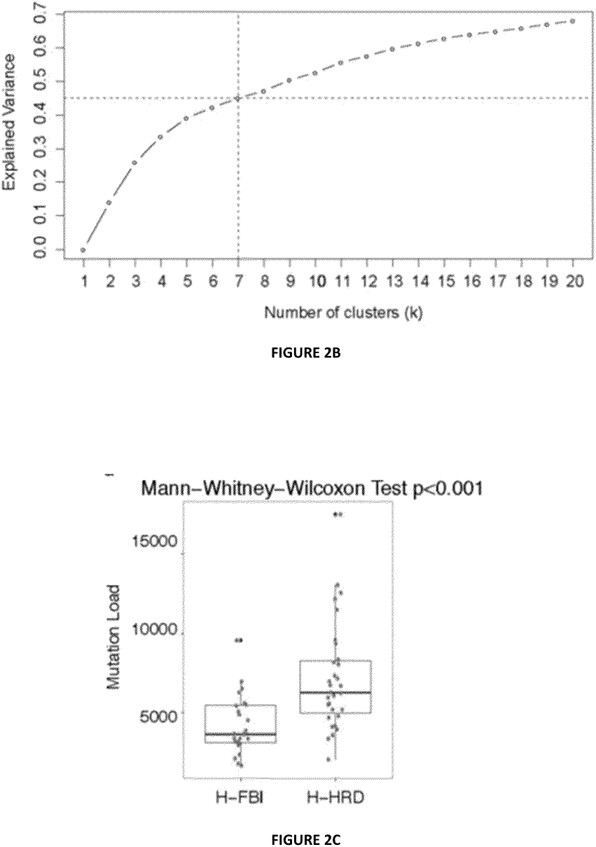

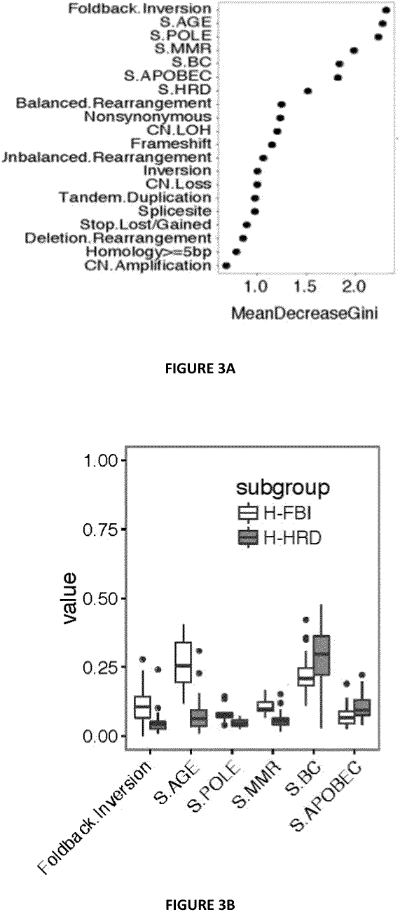

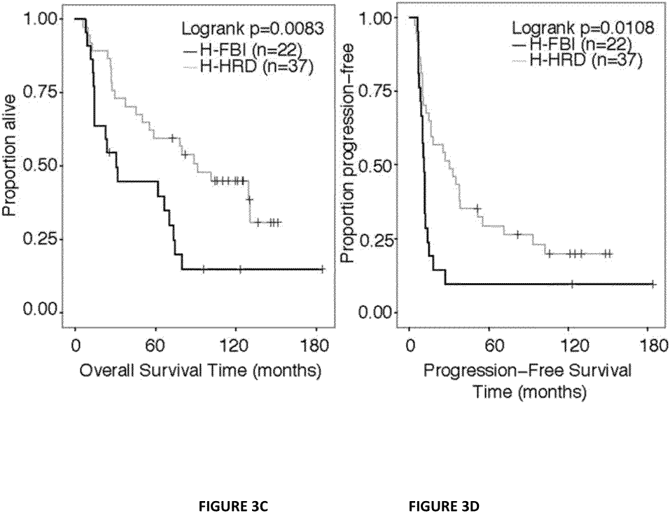

Ab-initio hierarchical clustering of the 133 ovarian cancer patients based on the 20 genomic features revealed seven major subgroups (FIG. 2A), with the optimal number of groups determined through marginal gain of explained variance (FIG. 2B). Linear discriminant analysis identified the dominant feature(s) distinguishing the tumours in each group (FIG. 1). Each of the three main histotypes stratified into subgroups, while all GCTs grouped together. The seven groups were characterized as follows: G-BC: GCT tumours with mutation signature S.BC (associated with breast cancer and medulloblastoma); E-MSI: MSI ENOC tumours characterized by mutation signature S.MMR (reflective of mismatch repair deficiency); Mixture: HGSC, CCOC and ENOC cases without obvious discriminant features; C-APOBEC: CCOC cases characterized by mutation signature S.APOBEC (attributed to activity of the AID/APOBEC family of cytidine deaminases); C-AGE: CCOC cases characterized by mutation signature S.AGE (associated with age at diagnosis); H-FBI: HGSC cases with high prevalence of fold-back inversion structural variations; and H-HRD: HGSC with prevalence of duplications or deletion rearrangements and mutation signature S.HRD (reflective of homologous recombination deficiency). Similar results were obtained whether clustering was performed on all histotypes together or when HGSC (FIGS. 3A-D) and endometriosis-associated samples (CCOC and ENOC) were clustered independently.

### Example 3 - A Novel Subgroup of HGSC Characterized by Fold-Back Inversions

Two subgroups comprised primarily of HGSC cases: H-FBI (n = 24, 41% of HGSC) and H-HRD (n = 31, 53%; Table 2). Table 2 shows the integration of genomic features stratifies ovarian cancer patients, with respect to the contribution of genomic subgroup memberships in each histotype. The number (n) and proportion (%) of samples from each subgroup are shown. Enrichment of cases (per histotype) in subgroups was assessed using Fisher’s exact test (corresponding p-values shown for significant enrichment p <0.01

TABLE 2

ENOC

| Subgroup 1 | % | N | P-value |
|---|---|---|---|
| G-BC | 7 | 2 | |
| E-MSI | 28 | 8 | P<0.001 |
| Mixture | 14 | 4 | |
| C-APOBEC | 7 | 2 | |
| C-AGE | 24 | 7 | |
| H-FBI | 7 | 2 | |
| H-HRD | 14 | 4 | |
| Subgroup | % | N | P-value |
| G-BC | 3 | 1 | |
| E-MSI | 0 | 0 | |
| Mixture | 23 | 8 | |
| C-APOBEC | 26 | 9 | P<0.001 |
| C-AGE | 40 | 14 | P<0.001 |
| H-FBI | 9 | 3 | |
| H-HRD | 0 | 0 | |
| Subgroup | % | N | P-value |
| G-BC | 0 | 0 | |
| E-MSI | 0 | 0 | |
| Mixture | 3 | 2 | |
| C-APOBEC | 2 | 1 | |
| C-AGE | 2 | 1 | |
| H-FBI | 41 | 24 | P<0.001 |
| H-HRD | 53 | 31 | P<0.001 |
| Subgroup | % | N | P-value |
| G-BC | 100 | 10 | P<0.001 |
| E-MSI | 0 | | |
| Mixture | 0 | | |
| C-APOBEC | 0 | | |
| C-AGE | 0 | | |
| H-FBI | 0 | | |
| H-HRD | 0 | | |

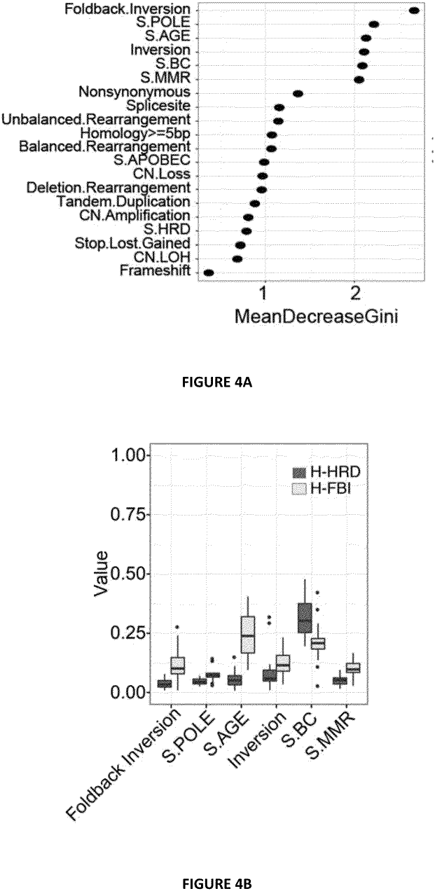

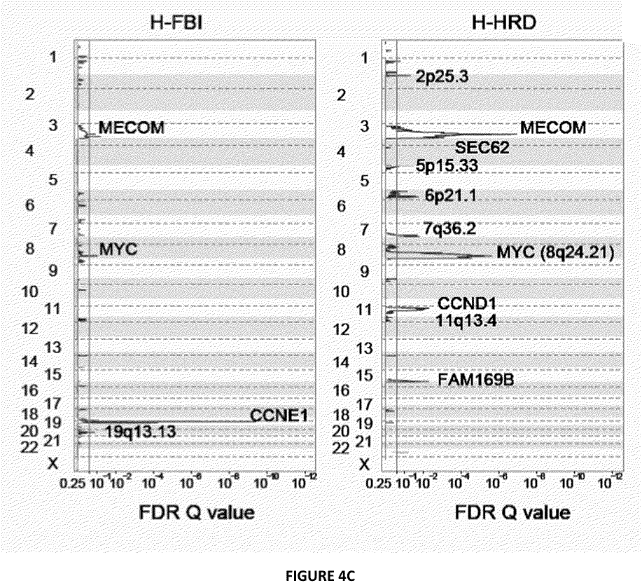

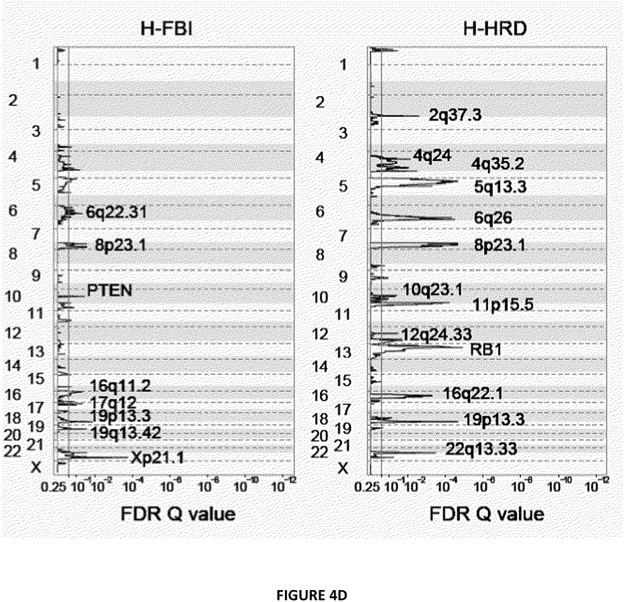

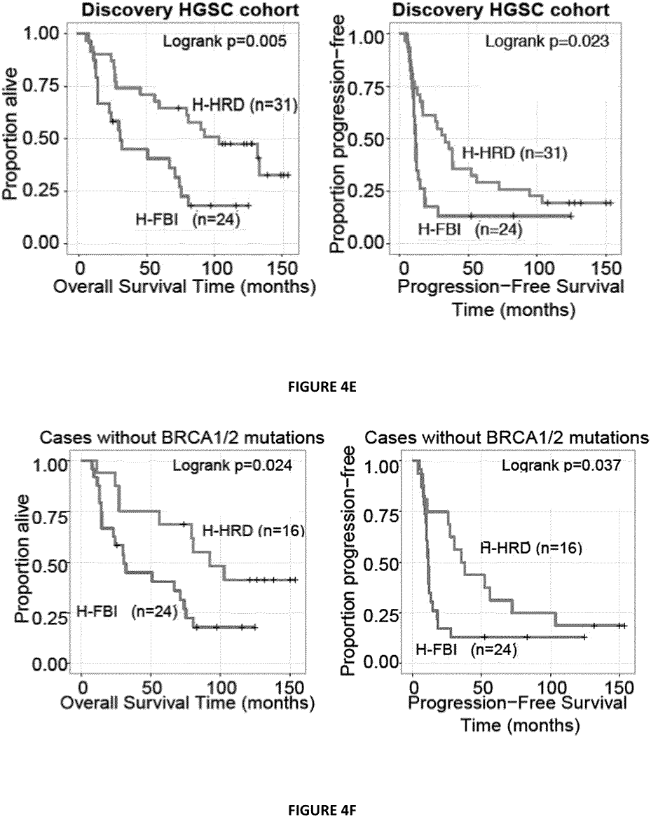

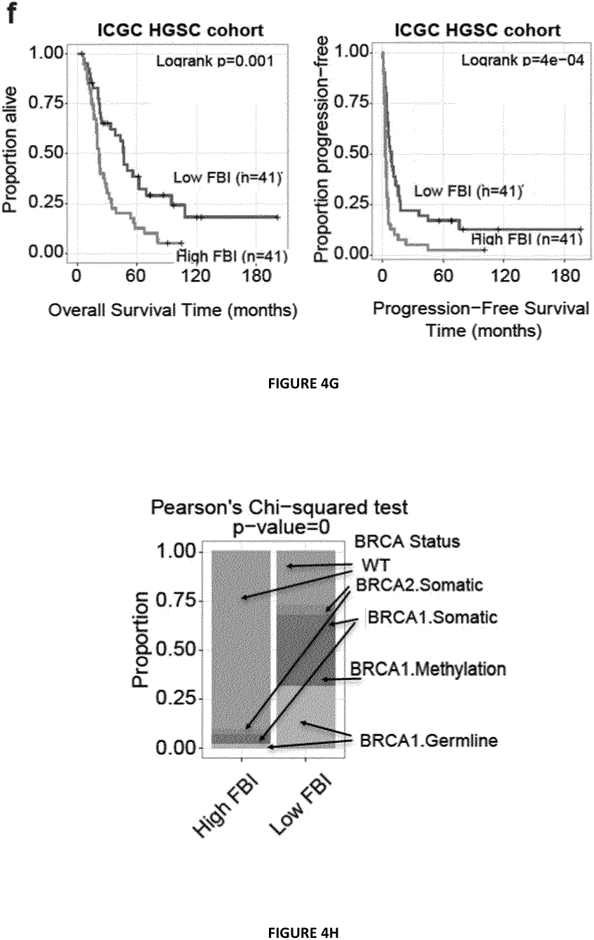

Relative importance analysis of H-FBI and H-HRD highlighted structural variation patterns, dominated by fold-back inversions, as discriminant features between the HGSC groups; this was corroborated by statistically higher distributions over the proportion of fold-back inversions over all SVs in H-FBI relative to H-HRD (mean 0.12 vs. 0.04 of SVs, P-value <0.0001; FIGS. 4A-D). Further differences were found in mutation signatures: H-HRD exhibited higher C_{S.H} _{RD} than H-FBI (mean 0.42 vs. 0.29, P-value <0.0001), while C_{S.AGE} was higher in H-FBI (mean 0.25 vs. 0.06 in H-HRD, P-value <0.000), and H-HRD exhibited higher overall mutation load (FIG. 4D).

Gene-based co-association revealed tumours harbouring BRCA1 somatic (n = 1) or germline mutations (n = 8), methylation of BRCA1 promoter (n = 3), BRCA2 somatic (n = 1) or germline mutations (n = 5) in the H-HRD group (FIG. 1 and Table 2). Analysis of a set of 61 genes related to homologous recombination pathways identified variants in 31 HR genes (point mutations in 18 genes and gene breakages in 20 genes), including group-specific RAD51B gene breakage in H-FBI cases (n=7 of 24, 29%) and NF1 disruption in H-HRD cases (n = 7 of 31, 23%). Statistically overrepresented focal copy number amplification of CCNE1 (19q21) was observed in H-FBI, whereas H-HRD implicated MECOM (3q26.2), MYC (8q24.21) and CCND1 (11q13.3) as over-represented regions of focal copy number amplification (FIG. 2B). Focal loss in PTEN (1 0q23.31) was uniquely identified in H-FBI and RB1 focal copy number deletion (FDR Q value <0.0001, n = 29, 94% of samples) was significant in H-HRD (FIG. 2C). In aggregate, these results identify distinct biological subgroups within HGSC, separated by mutational processes, in particular fold-back inversion events. Co-associated genomic disruptions of specific genes and pathways further implicates divergent biological properties of H-FBI and H-HRD cases.

### Example 4 - Fold-back Inversions Associate With Inferior Prognosis in HGSC

The survival response to uniform, platinum-based chemotherapy in the two HGSC subgroups was compared. The overall (OS) and progression-free survival (PFS) of the H-FBI subgroup were poor relative to H-HRD (logrank p-values ₌ 0.0053 and 0.0232; FIG. 4E). The outcome associations were independent of BRCA status, established by excluding the 15 cases with BRCA½ somatic or germline mutations and re-computing the survival curves (FIG. 4F; logrank p-values = 0.024 and 0.037). The fold-back inversion proportion was isolated as a feature and stratified cases into two groups (High FBI and Low FBI) based on the median value. Both OS and PFS were significantly worse for the High FBI group, establishing that the fold-back inversion proportion could be used as a single prognostic feature (logrank p-values = 0.0187 and 0.0286; FIG. 5A).

This approach was validated in an external cohort (International Cancer Genome Consortium (ICGC; Ha, G. et al., 2014). The 82 HGSC ICGC primary tumours were stratified into two equal-sized groups according to the prevalence of fold-back inversions. The ICGC data was sequenced independently, structural variations were predicted with an orthogonal computational method, and the cohort was not controlled for uniform treatment protocols. Despite these variations, the ICGC group with high prevalence of fold-back inversions (High FBI, n = 41) associated with poor outcome (Low FBI, n = 41; logrank p-values <0.0001 for both OS and PFS; FIG. 4G). Enrichment of BRCA½ mutants was observed in the Low FBI group (Chi-squared test P-value = 0; FIG. 4H), including cases with BRCA1 somatic (n = 3 out of 5, 60%) or germline mutations (n = 13 out of 14, 93%), methylation of BRCA1 promoter (n = 12), or BRCA2 somatic (n = 2 out of 3, 67%), consistent with the observation of cases with BRCA½ mutations prevalent in the H-HRD subgroup of the discovery cohort. Notably, H-HRD and H-FBI were not associated with previously described molecular subtypes (Ahmed, A. A. et al., 2010; Ding, J. et al., 2012) in the discovery cohort (Chi-squared test P-value = 0.6129) nor in the ICGC cohort (Chi-squared test P-value = 0.0928, FIG. 4I), indicating fold-back inversion is independent of gene expression-based subgroups.

### Example 5 - Fold-Back Inversions Co-Localize With High-Level Amplifications

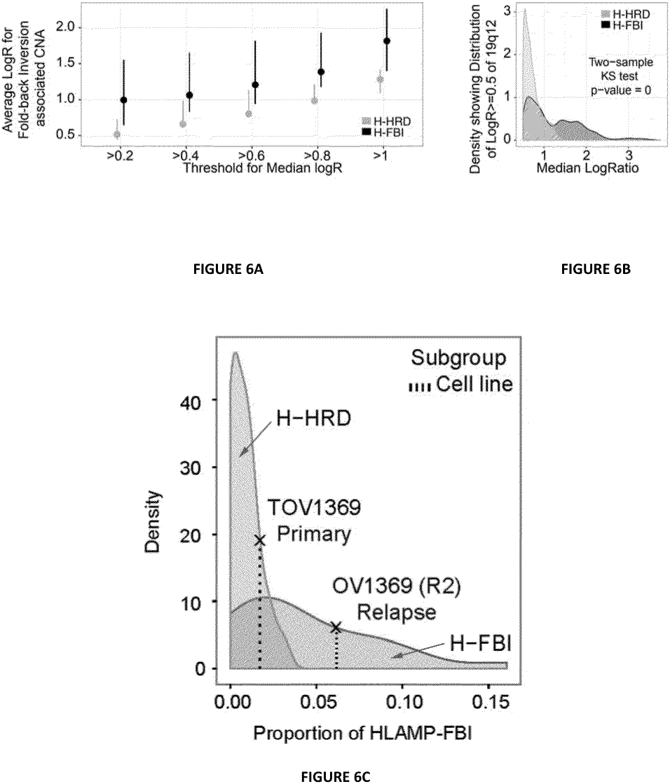

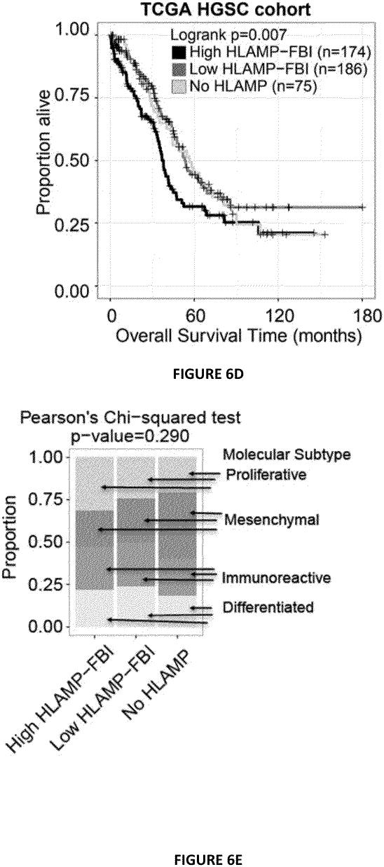

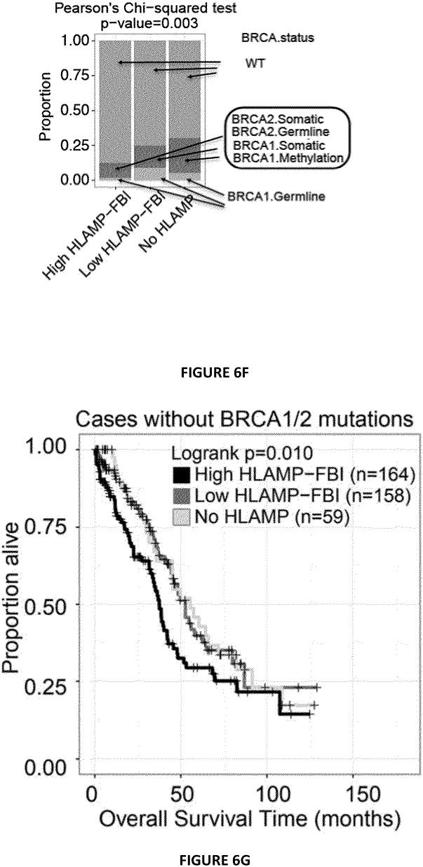

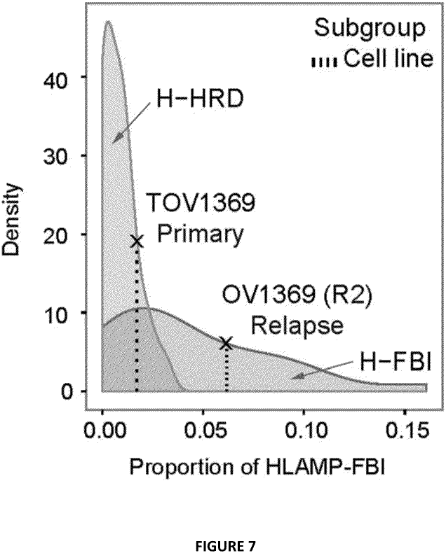

Fold-back inversions in HGSC tumour genomes were characterized by short breakage distances between two breakpoints (median = 2329 bp; FIG. 5B). Two homologous short sequences indicative of microhomology, were observed on both strands at the breakpoints of fold-back inversion, suggesting MMEJ DNA repair mechanisms (Saunders, C. T. et al., 2012; Cingolani, P. et al., 2012) could be operating in these tumours. As fold-back inversions are a consequence of breakage fusion bridge cycles leading to amplification, high-level amplifications (HLAMP) associated with fold-back inversions in H-HRD and H-FBI cases were examined. The average log ratio (LogR) of the CNA profiles at the breakpoints of fold-back inversions between the two subgroups were examined at increasing levels of LogR (FIG. 6A). Significantly greater LogR in H-FBI was found than in H-HRD at high-level amplification events (median LogR >1; Mann-Whitney-Wilcoxon Test adjusted P-value = 0.0099). Regions of significant recurrent amplifications were examined across H-HRD and H-FBI HGSCs (FIG. 6B) and structural rearrangement types associated with these events were computed. The most prominent signal from recurrent amplifications was found in the 19q21 regions harbouring CCNE1. The distribution of LogR in 19q21 events was accordingly higher in the H-FBI group relative to H-HRD (FIG. 6B). Furthermore, amplification of 19q21 events showed a co-occurrence of copy number state change and breakpoints of fold-back inversions at the focal aberrant regions of CCNE1 in H-FBI cases and in ICGC High FBI cases. High-level amplifications at 12p12.1 (KRAS) and 8q24.21 (MYC) also showed precise localization of fold-back inversions at large copy number transition points of high level amplification events in H-FBI discovery cohort and High FBI ICGC cases. In addition, the genomes and identified fold-back inversion associated high-level amplifications in a pair of previously characterized HGSC cell lines (TOV1369 and OV1369 (R2)) derived from two, temporally sampled primary and relapse specimens from the same patient pre- and post-chemotherapy were sequenced (Lawrence, M. S. et al., 2013). The proportion of fold-back inversion co-localized with HLAMPs of the primary and relapse cell lines fell squarely within the H-HRD and H-FBI distributions from the discovery cohort (FIG. 7), commensurate with a shift pre-and post-chemotherapy in IC50 values (Carboplatin (avg+/-SD): 5.64+/-1.29 for TOV1369 and 9.8+/-1.00 for OV1369 (R2)).

The TOV1369 and OV1369 (R2) cell lines were profiled for carboplatin response and showed a shift pre- and post-chemotherapy in IC50 values (Carboplatin (avg+/-SD): 5.64+/-1.29 for TOV1369 and 9.8+/-1.00 for OV1369 (R2) (Le′tourneau, I. J. et al., 201) suggesting acquired resistance over time. The genomes were sequenced and fold-back inversion associated high-level amplifications in each cell line were identified. An overall increase in events across the genome in the relapse-derived sample was observed. The proportion of fold-back inversion co-localized with HLAMPs of the primary and relapse cell lines falls squarely within the H-HRD and H-FBI distributions from the discovery cohort (FIG. 6C), consistent with a shift in genomic properties. Specific examples of acquisition (or selection) of events could be seen on chr5, chr6 and chr18, amongst others. In context of the patient samples from the discovery and ICGC cohorts, these results suggest progression may be related to accumulation of fold-back inversion events in the presence of chemotherapy and point to mechanistic relevance of co-localized fold-back inversions and HLAMPs.

### Example 6 - Prognostic Stratification of HGSC by Fold-Back Inversion Associated High-Level Amplifications

Prognostic relevance of the association of fold-back inversion and high-level amplification was tested in the Cancer Genome Atlas (TCGA) Ovarian serous cystadenocarcinoma (OV) cohort (Cancer Genome Atlas Research Network, 2011. Exome capture sequence and outcome data were available for 435 patients. Copy number profiles were analyzed to identify regions harbouring HLAMPs (copy number LogR ≥ 1) for each patient (excluding cases (n = 75) with zero copy number events with LogR ≥ 1) and profiled structural variations in these regions. The subset of amplification events co-localized with fold-back inversion. Average LogR was then computed for each case over amplified regions associated with fold-back inversions, which as a score stratified 360 cases into two groups. Cases prevalent in fold-back inversion associated amplifications with high LogR (Average LogR > median score; n = 174) were labeled as “FBI-AMP High”, with the remainder (Average LogR ≤ median score; n = 186) as “FBI-AMP Low”. Overall survival for FBI-AMP High cases was poor relative to FBI-AMP Low and No AMP (log-rank test p-value = 0.007; FIG. 6C). Consistent with the discovery HGSC and the ICGC cohorts, no association was observed between previously defined molecular subtypes (Cancer Genome Atlas Research Network, 2011; Tothill, R. W. et al., 2008) and the three groups (Chi-squared test p-value = 0.290). Enrichment of BRCA mutants was identified in No AMP and FBI-AMP Low (Chi-squared test p-value = 0.003; FIG. 6D). However, as for the discovery cohort, excluding BRCA mutants did not impact overall survival differences between the three groups (log-rank test p-value = 0.010; FIG. 6E), establishing in a large series that fold-back inversions associated with amplification events transcend both BRCA mutation status and gene expression based molecular subgroups.

Stratification of ovarian carcinomas by genomic characteristics could provide an aetiologic model for ovarian cancer histotypes, and a framework with which to research and treat ovarian cancer patients. FIG. 9 shows a summary of the findings with specific illustrative examples, depicting divisions within histotypes as specific pathways to tumour progression which have potential implications on therapeutic options. HGSC are thought to originate in the fallopian tube with early evolutionary acquisition of TP53 mutation and LOH of chromosome 17 (Alexandrov, L. B. et al., (2013); Layer, R. M., et al., 2014). Our results suggest a subsequent divergence whereby tumours acquire contrasting properties of double strand break DNA repair processes. In summary, some cases (H-HRD) exhibited tandem duplication- and/or unbalanced rearrangement-induced amplifications and had increased proportions of deletions and LOH across their genomes, while another distinct group (H-FBI) exhibited fold-back inversions co-associated with high-level amplifications. As fold-back inversions with microhomology are reflective of active MMEJ processes, we suggest that these HGSC tumours may have increased capacity to repair events induced by genotoxic chemotherapy. As such, these cancers may not be responsive to PARP inhibitors and, in the independent cohorts presented here, show evidence of poor response to cisplatin.

Poor survival outcome in cases with fold-back inversions was seen in three independent cohorts and by three independent methods. This was true of all non-BRCA mutant cases. This may indicate that BRCA testing alone is insufficient as a biomarker for directing patients onto specific interventions. The genome itself may reflect the DNA repair and mutational processes that were active in its evolutionary history, providing highly discriminant features and potent signals of biological variation for patient stratification.

Several potential therapeutic opportunities for ovarian cancer across the spectrum of histotypes are implicated by these findings. Recent progress in elucidating mechanisms that activate and repress MMEJ (Chiang, C. et al., 2015) and dependency of HR deficient cells on MMEJ (McPherson, A. et al., 2012) implicate Polθ as a promising therapeutic target for HGSC. The results presented suggest that fold-back inversion attributes may signal a vulnerability by targeting MMEJ through Polθ inhibition. Specifically, the fold-back inversion mutation signature reported here could provide a critical link to identifying patients that may benefit from targeting MMEJ. Potential therapeutic options for targeting MMEJ could include sensitization to cisplatin or PARPi, or through the use of alternative therapeutic modalities.

### Example 7 - Multiple Mutational Signatures Stratify Endometriosis-Associated Ovarian Cancers

In contrast to HGSC, endometriosis-associated cancers (CCOC and ENOC) were primarily stratified on the basis of SNV mutational signatures (FIGS. 8A-D). Two major subgroups of CCOC were identified; one group (C-APOBEC, n = 9, 26%) characterized by S.APOBEC mutational signature (mean C_{S.APOBEC} = 0.55 vs. 0.10, P-value = 0.02) while the other group (C-AGE, n = 14, 40%) characterized by S.AGE (mean C_{S.AGE} = 0.40 vs 0.20, P-value = 0.03; FIGS. 8E-F). Kataegis analysis (Alexandrov, L. B. et al., 2013; Nik-Zainal, S. et al., 2012) detected the regions of localized hypermutations with clusters of C >T and C >G in eight CCOC cases, five of which (55.6%) were members of C- APOBEC. No statistical difference in the prevalence of either ARID1A or PIK3CA mutations between C-APOBEC and C-AGE (n = 8/9, 89% vs. n = 12/14, 86%) was observed. 20q13.2 HLAMP encoding ZNF217was identified in both C-APOBEC (n = 3, 33%) and C-AGE (n = 8, 57%) (FDR q values = 0.01 and <0.0001, respectively). Focally deleted regions (FDR q values <0.1) including 1p35.3 encoding ARID1A (n = 10, 29%), 8p22 (n = 8, 23%) and 18q22.2 (n = 16, 46%) was observed in CCOC tumours.

For ENOC tumours, a clear subgroup (labeled as E-MSI) was delineated by S.MMR and corresponded directly to the MSI positive cases (FIGS. 8C-D and 8G-H). The remaining MSS tumours were distributed across the remaining six groups, indicating a high degree of heterogeneity and without informative discriminant genomic features. Across all ENOC tumours, gene-based analysis recapitulated known mutation patterns. Homozygous deletion in PTEN (n = 3) was mutually exclusive to PIK3CA mutation in ENOC tumours, and CTNNB1 (n = 10) mutation was mutually exclusive to KRAS (n = 10) mutation. TP53 was frequently mutated in MSS cases (n = 8 of 20, 40%) while RPL22 was frequently mutated in E-MSI cases (n = 4 of 8, 50%). Significantly more frameshifting indels in E-MSI cases (on average 0.2% vs 0.08% of all mutations) were found compared to MSS ENOC tumours (Student’s t-test, adjusted p-value = 0.005), consistent with MMR deficiency resulting in an increased prevalence of small indels at nucleotide repeats (Alexandrov, L. B. et al., 2013). The focal recurrent somatic CNAs in ENOC tumours were examined and focally amplified regions of 3q26.2 encoding MECOM, 8q24.21 encoding MYC and deleted regions of 10q23.31 encoded PTEN in MSS cases were identified. Consistent with known depletion of copy number events in hyper-mutated cancers, no focal CNAs were observed in MSI cases. Finally, the number of immunogenic epitopes generated in the ENOC patients was estimated and significantly higher counts in MSI compared with MSS cases were observed (Kruskal-Wallis rank sum test Chi-squared p-value = 0.0023), suggesting higher rates of neoantigen generation in ENOC MSI cases.

Endometriosis-associated tumours share a common etiologic origin and often harbour ARID1A and PIK3CA mutations. However, these cancers grouped according to non-overlapping mutational processes. Approximately one third of ENOC patients exhibited microsatellite instability (E-MSI) with an accompanying mutation signature reflective of mismatch repair deficiency, and a high proportion of frameshifting indels. These cases harboured approximately 10-fold more coding mutations and showed evidence of generating neoantigens at a higher rate than other ENOC tumours. Recent successful application of the PD-1 blockade compound pembrolizumab in mismatch repair deficient colorectal and non-colorectal cancers (Le, D. T. et al.; 2015) signals that MMR deficient ENOC cancers could be candidates for immunotherapy.

Twenty six percent of CCOC cases exhibited a mutational profile consistent with APOBEC- related mutational processes (C-APOBEC). APOBEC signature association was independent of ARID1A and PIK3CA mutation status suggesting these cases may have a unique aetiology unrelated to known driver mutation status. APOBEC-mediated deamination has been implicated as a clonal diversity-generating mechanism. As such, the APOBEC mutational process has been proposed as a therapeutic target in order to prevent ongoing clonal evolution in disease progression (McPherson, A. et al., 2011). Our results identify a subset of CCOC that could be candidates for APOBEC targeting. Although ENOC and CCOC share aetiologic origin in endometriosis, their SNV mutation spectra indicate divergence along distinct mutational processes and pathways within and between cancers that share histologic characteristics.

### Example 8 - Breast Cancers

Stratification of 63 triple negative breast cancers indicated patterns similar to those found for ovarian cancers. More specifically, in a study showing the relative signature activity of each of the cases in which both single nucleotide variant (SNV) and structural variation (SV) signatures were represented, SV-1 (a foldback inversion signature) and SNV-3 (HRD signature) bearing cases were nearly exclusive to each other and exhibited similar stratifications seen in high grade serous ovarian cancer.

All citations are hereby incorporated by reference.

The present invention has been described with regard to one or more embodiments. However, it will be apparent to persons skilled in the art that a number of variations and modifications can be made without departing from the scope of the invention as defined in the claims.

### REFERENCES

1. Kobel, M. et al. Ovarian carcinoma subtypes are different diseases: implications for biomarker studies. PLoS medicine 5, e232 (2008).

2. Vaughan, S. et al. Rethinking ovarian cancer: recommendations for improving outcomes. Nature Reviews Cancer 11, 719-725 (2011).

3. Risch, H. et al. Population BRCA1 and BRCA2 mutation frequencies and cancer penetrances: a kin-cohort study in Ontario, Canada. J Natl Cancer Inst 98, 1694-1706 (2006).

4. Alsop, K. et al. Brca mutation frequency and patterns of treatment response in BRCA mutation- positive women with ovarian cancer: a report from the Australian ovarian cancer study group. Journal of Clinical Oncology 30, 2654-2663 (2012).

5. Berns, E. M. & Bowtell, D. D. The changing view of high-grade serous ovarian cancer. Cancer research 72, 2701-2704 (2012).

6. Anglesio, M. S., Carey, M. S., Kobel, M., MacKay, H. & Huntsman, D. G. Clear cell carcinoma of the ovary: a report from the first ovarian clear cell symposium, june 24th, 2010. Gynecologic oncology 121, 407-415 (2011).

7. Munksgaard, P. S. & Blaakaer, J. The association between endometriosis and ovarian cancer: a review of histological, genetic and molecular alterations. Gynecologic oncology 124, 164-169 (2012).

8. Ahmed, A. A. et al. Driver mutations in TP53 are ubiquitous in high grade serous carcinoma of the ovary. The Journal of pathology 221, 49-56 (2010).

9. Cancer Genome Atlas Research Network. Integrated genomic analyses of ovarian carcinoma. Nature 474, 609-15 (2011).

10. Wiegand, K. C. et al. Arid1a mutations in endometriosis-associated ovarian carcinomas. The New England journal of medicine 363, 1532-1543 (2010).

11. Jones, S. et al. Frequent mutations of chromatin remodeling gene ARID1A in ovarian clear cell carcinoma. Science 330, 228-231 (2010).

12. Obata, K. et al. Frequent PTEN/MMAC mutations in endometrioid but not serous or mucinous epithelial ovarian tumors. Cancer research 58, 2095-2097 (1998).

13. Wu, R., Zhai, Y., Fearon, E. R. & Cho, K. R. Diverse mechanisms of β-catenin deregulation in ovarian endometrioid adenocarcinomas. Cancer research 61, 8247-8255 (2001).

14. Campbell, I. G. et al. Mutation of the PIK3CA gene in ovarian and breast cancer. Cancer research 64, 7678-7681 (2004).

15. Kuo, K.-T. et al. Frequent activating mutations of PIK3CA in ovarian clear cell carcinoma. The American journal of pathology 174, 1597-1601 (2009).

16. Kurman, R. J. & Shih, I.-M. Molecular pathogenesis and extraovarian origin of epithelial ovarian cancer shifting the paradigm. Human pathology 42, 918-931 (2011).

17. McConechy, M. K. et al. Subtype-specific mutation of PPP2R1a in endometrial and ovarian carcinomas. The Journal of pathology 223, 567-573 (2011).

18. Nissenblatt, M. Endometriosis-associated ovarian carcinomas. N. Engl. J. Med 364, 482-483 (2011).

19. Wu, R.-C. et al. Frequent somatic mutations of the telomerase reverse transcriptase promoter in ovarian clear cell carcinoma but not in other major types of gynaecological malignancy. The Journal of pathology 232, 473-481 (2014).

20. Niskakoski, A. et al. Distinct molecular profiles in lynch syndrome-associated and sporadic ovarian carcinomas. International Journal of Cancer 133, 2596-2608 (2013).

21. Shah, S. P. et al. Mutation of FOXL2 in granulosa-cell tumors of the ovary. New England Journal of Medicine 360, 2719-2729 (2009).

22. Piccart, M. J. et al. Randomized intergroup trial of cisplatin-paclitaxel versus cisplatin-cyclophosphamide in women with advanced epithelial ovarian cancer: three-year results. J Natl Cancer Inst 92, 699-708 (2000).

23. RAUH-HAIN, J. & Penson, R. Potential benefit of Sunitinib in recurrent and refractory ovarian clear cell adenocarcinoma. International Journal of Gynecological Cancer 18, 934-936 (2008).

24. McAlpine, J. N. et al. HER2 overexpression and amplification is present in a subset of ovarian mucinous carcinomas and can be targeted with trastuzumab therapy. BMC cancer 9, 1 (2009).

25. Anglesio, M. S. et al. IL6-STAT3-HIF signaling and therapeutic response to the angiogenesis inhibitor sunitinib in ovarian clear cell cancer. Clinical cancer research 17, 2538-2548 (2011).

26. Farley, J. H., Gibson, S. J. & Monk, B. J. American society of clinical oncology 2012 annual meeting update: Summary of selected gynecologic cancer abstracts. Gynecologic oncology 126, 319-324 (2012).

27. Anglesio, M. S. et al. Molecular characterization of mucinous ovarian tumours supports a stratified treatment approach with HER2 targeting in 19% of carcinomas. The Journal of pathology 229, 111-120 (2013).

28. Ledermann, J. et al. Olaparib maintenance therapy in platinum-sensitive relapsed ovarian cancer. The New England journal of medicine 366, 1382-1392 (2012).

29. Ledermann, J. et al. Olaparib maintenance therapy in patients with platinum-sensitive relapsed serous ovarian cancer: a preplanned retrospective analysis of outcomes by BRCA status in a randomised phase 2 trial. The Lancet. Oncology 15, 852-861 (2014).

30. Mirza, M. R. et al. Niraparib maintenance therapy in platinum-sensitive, recurrent ovarian cancer. The New England journal of medicine 375, 2154-2164 (2016).

31. Swisher, E. M. et al. Rucaparib in relapsed, platinum-sensitive high-grade ovarian carcinoma (ARIEL2 part 1): an international, multicentre, open-label, phase 2 trial. The Lancet. Oncology 18, 75-87 (2017).

32. Alexandrov, L. B. et al. Signatures of mutational processes in human cancer. Nature 500, 415-21 (2013).

33. Nik-Zainal, S. et al. Mutational processes molding the genomes of 21 breast cancers. Cell 149, 979-93 (2012).

34. Campbell, P. J. et al. The patterns and dynamics of genomic instability in metastatic pancreatic cancer. Nature 467, 1109-1113 (2010).

35. Sudmant, P. H. et al. An integrated map of structural variation in 2,504 human genomes. Nature 526, 75-81 (2015).

36. Ng, C. K. Y. et al. The role of tandem duplicator phenotype in tumour evolution in high-grade serous ovarian cancer. The Journal of pathology 226, 703-712 (2012).

37. Sasaki, S. et al. Molecular processes of chromosome 9p21 deletions in human cancers. Oncogene 22, 3792-3798 (2003).

38. Yang, L. et al. Diverse mechanisms of somatic structural variations in human cancer genomes. Cell 153, 919-929 (2013).

39. Hermetz, K. E. et al. Large inverted duplications in the human genome form via a fold-back mechanism. PLoS Genet 10, e1004139 (2014).

40. Ha, G. et al. TITAN: inference of copy number architectures in clonal cell populations from tumor whole-genome sequence data. Genome Res 24, 1881-93 (2014).

41. Ding, J. et al. Feature-based classifiers for somatic mutation detection in tumour-normal paired sequencing data. Bioinformatics 28, 167-175 (2012).

42. Saunders, C. T. et al. Strelka: accurate somatic small-variant calling from sequenced tumor-normal sample pairs. Bioinformatics 28, 1811-1817 (2012).

43. Cingolani, P. et al. A program for annotating and predicting the effects of single nucleotide polymorphisms, snpeff: Snps in the genome of drosophila melanogaster strain w1118; iso-2; iso-3. Fly 6, 80-92 (2012).

44. Lawrence, M. S. et al. Mutational heterogeneity in cancer and the search for new cancer- associated genes. Nature 499, 214-218 (2013).

45. Alexandrov, L. B. et al. Signatures of mutational processes in human cancer. Nature 500, 415-21 (2013).

46. Layer, R. M., Chiang, C., Quinlan, A. R. & Hall, I. M. Lumpy: a probabilistic framework for structural variant discovery. Genome biology 15, 1 (2014).

47. Chiang, C. et al. Speedseq: ultra-fast personal genome analysis and interpretation. Nature methods (2015).

48. McPherson, A. et al. nFuse: discovery of complex genomic rearrangements in cancer using high-throughput sequencing. Genome Res 22, 2250-61 (2012).

49. Gunawardana, J. et al. Recurrent somatic mutations of ptpn1 in primary mediastinal b cell lymphoma and hodgkin lymphoma. Nature genetics 46, 329-335 (2014).

50. McPherson, A. et al. defuse: an algorithm for gene fusion discovery in tumor rna-seq data. PLoS Comput Biol 7, e1 001138 (2011).

51. Hormozdiari, F. et al. Next-generation variationhunter: combinatorial algorithms for transpo- son insertion discovery. Bioinformatics 26, i350-i357 (2010).

52. Patch, A.-M. et al. Whole-genome characterization of chemoresistant ovarian cancer. Nature 521(7553), 489-94 (2015).

53. Abo, R. P. et al. Breakmer: detection of structural variation in targeted massively parallel sequencing data using kmers. Nucleic acids research 43, e19-e19 (2015).

54. Le ́tourneau, I. J. et al. Derivation and characterization of matched cell lines from primary and recurrent serous ovarian cancer. BMC cancer 12, 1 (2012).

55. Fleury, H. et al. Cumulative defects in dna repair pathways drive the parp inhibitor response in high-grade serous epithelial ovarian cancer cell lines. Oncotarget 5 (2016).

56. Fleury, H. et al. Novel high-grade serous epithelial ovarian cancer cell lines that reflect the molecular diversity of both the sporadic and hereditary disease. Genes & cancer 6, 378 (2015).

57. Ha, G. et al. Integrative analysis of genome-wide loss of heterozygosity and mono-allelic expression at nucleotide resolution reveals disrupted pathways in triple negative breast cancer. Genome Research (2012).

58. Eirew, P. et al. Dynamics of genomic clones in breast cancer patient xenografts at single-cell resolution. Nature (2014).

59. McPherson, A. et al. Divergent modes of clonal spread and intraperitoneal mixing in high- grade serous ovarian cancer. Nature genetics (2016).

60. Langmead, B. & Salzberg, S. L. Fast gapped-read alignment with bowtie 2. Nature methods 9, 357-359 (2012).

61. McKenna, A. et al. The genome analysis toolkit: a mapreduce framework for analyzing next- generation dna sequencing data. Genome research 20, 1297-1303 (2010).

62. DePristo, M. A. et al. A framework for variation discovery and genotyping using next- generation dna sequencing data. Nature genetics 43, 491-498 (2011).

63. Auwera, G. A. et al. From fastq data to high-confidence variant calls: the genome analysis toolkit best practices pipeline. Current protocols in bioinformatics 11-10 (2013).

64. Shah, S. P. et al. The clonal and mutational evolution spectrum of primary triple-negative breast cancers. Nature 486, 395-399 (2012).

65. Shah, S. et al. Mutational evolution in a lobular breast tumour profiled at single nucleotide resolution. Nature 461, 809-813 (2009).

66. Kalloger, S. E. et al. Calculator for ovarian carcinoma subtype prediction. Modern Pathology 24, 512-521 (2011).

67. Anglesio, M. S., Carey, M. S., Ko ̈ bel, M., MacKay, H. & Huntsman, D. G. Clear cell car- cinoma of the ovary: a report from the first ovarian clear cell symposium, june 24th, 2010. Gynecologic oncology 121, 407-415 (2011).

68. Madore, J. et al. Characterization of the molecular differences between ovarian endometrioid carcinoma and ovarian serous carcinoma. The Journal of pathology 220, 392-400 (2010).

69. Ko¨bel, M. et al. Igf2bp3 (imp3) expression is a marker of unfavorable prognosis in ovarian carcinoma of clear cell subtype. Modern Pathology 22, 469-475 (2009a).

70. Ko¨bel, M. et al. A limited panel of immunomarkers can reliably distinguish between clear cell and high-grade serous carcinoma of the ovary. The American journal of surgical pathology 33, 14-21 (2009b).

71. Ko¨bel, M. et al. Ovarian carcinoma subtypes are different diseases: implications for biomarker studies. PLoS Med 5, e232 (2008).

72. Helland, A ̊. et al. Deregulation of mycn, lin28b and let7 in a molecular subtype of aggressive high-grade serous ovarian cancers. PloS one 6, e18064 (2011).

73. Anglesio, M. S. et al. 116-stat3-hif signaling and therapeutic response to the angiogenesis inhibitor sunitinib in ovarian clear cell cancer. Clinical cancer research 17, 2538-2548 (2011).

74. Anglesio, M. S. et al. Mutation of erbb2 provides a novel alternative mechanism for the ubiquitous activation of ras-mapk in ovarian serous low malignant potential tumors. Molecular Cancer Research 6, 1678-1690 (2008).

75. Tothill, R. W. et al. Novel molecular subtypes of serous and endometrioid ovarian cancer linked to clinical outcome. Clinical Cancer Research 14, 5198-5208 (2008).

76. Cancer Genome Atlas Research Network. Integrated genomic analyses of ovarian carcinoma. Nature 474, 609-15 (2011).

77. Ramakrishna, M. et al. Identification of candidate growth promoting genes in ovarian cancer through integrated copy number and expression analysis. PloS one 5, e9983 (2010).

78. Hendrix, N. D. et al. Fibroblast growth factor 9 has oncogenic activity and is a downstream target of wnt signaling in ovarian endometrioid adenocarcinomas. Cancer research 66, 1354-1362 (2006).

79. Szolek, A. et al. Optitype: precision hla typing from next-generation sequencing data. Bioin- formatics 30, 3310-3316 (2014).

80. Hundal, J. et al. pvac-seq: A genome-guided in silico approach to identifying tumor neoanti- gens. Genome medicine 8, 1 (2016).

81. Nielsen, M. et al. Reliable prediction of t-cell epitopes using neural networks with novel sequence representations. Protein Science 12, 1007-1017 (2003).

82. Lundegaard, C. et al. Netmhc-3.0: accurate web accessible predictions of human, mouse and monkey mhc class i affinities for peptides of length 8-11. Nucleic acids research 36, W509- W512 (2008).

83. Lundegaard, C., Lund, O. & Nielsen, M. Accurate approximation method for prediction of class i mhc affinities for peptides of length 8, 10 and 11 using prediction tools trained on 9mers. Bioinformatics 24, 1397-1398 (2008).

84. Hoof, I., Peters, B., Buus, S. & Nielsen, M. Netmhcpan: Mhc class i binding prediction beyond hla-a and-b. Tissue Antigens (2008).

85. Nielsen, M. et al. Netmhcpan, a method for quantitative predictions of peptide binding to any hla-a and-b locus protein of known sequence. PloS one 2, e796 (2007).

86. Castel, S. E., Levy-Moonshine, A., Mohammadi, P., Banks, E. & Lappalainen, T. Tools and best practices for data processing in allelic expression analysis. Genome biology 16, 1 (2015).

87. Trapnell, C. et al. Transcript assembly and quantification by rna-seq reveals unannotated transcripts and isoform switching during cell differentiation. Nature biotechnology 28, 511-515 (2010).

88. Roberts, A., Trapnell, C., Donaghey, J., Rinn, J. L. & Pachter, L. Improving rna-seq expression estimates by correcting for fragment bias. Genome biology 12, 1 (2011a).

89. Roberts, A., Pimentel, H., Trapnell, C. & Pachter, L. Identification of novel transcripts in annotated genomes using rna-seq. Bioinformatics 27, 2325-2329 (2011b).

90. Trapnell, C. et al. Differential analysis of gene regulation at transcript resolution with rna-seq. Nature biotechnology 31, 46-53 (2013).

91. Le ́tourneau, I. J. et al. Derivation and characterization of matched cell lines from primary and recurrent serous ovarian cancer. BMC cancer 12, 1 (2012).

92. Le, D. T. et al. PD-1 blockade in tumors with mismatch-repair deficiency. The New England journal of medicine 372, 2509-2520 (2015).

93. Forbes et al Nucleic Acids Res. 2017 Jan 4;45(D1):D777-D783 (2017). PMID: 27899578

What is claimed is:

1. A method for determining the prognosis for a cancer patient in need thereof, the method comprising:
a) providing the genomic DNA sequence of a cancer sample from the patient;
b) detecting structural variation patterns in the genomic DNA sequence of the cancer sample; and
c) determining the prevalence of the structural variation patterns in the genomic DNA sequence of the cancer sample,
wherein a high level of fold-back inversions is indicative of a poor prognosis.

2. The method of claim 1 further comprising:
a) providing the genomic DNA sequence of a normal sample;
b) detecting structural variation patterns in the genomic DNA sequence of the normal sample; and
c) comparing the structural variation patterns in the genomic DNA sequence of the normal sample with those in the genomic DNA sequence of the cancer sample,
wherein the increased prevalence of fold-back inversions in the genomic DNA sequence of the cancer sample compared to the genomic DNA sequence of the normal sample is indicative of a poor prognosis.

3. The method of claim 1 or 2 further comprising detecting high-level amplifications in the genomic DNA sequence of the cancer sample, and the genomic DNA sequence of the normal sample, if present, wherein colocalization of the high-level amplifications and the fold-back inversions is indicative of a poor prognosis.

4. A method for the stratification of a cancer patient, the method comprising:
a) providing the genomic DNA sequence of a cancer sample from the patient;
b) detecting genomic features in the genomic DNA sequence of the cancer sample, the genomic features comprising single nucleotide variants, insertions/deletions, mutation signatures, and structural variants; and
c) stratifying the patient into a cancer subgroup based on the prevalence of one or more of the genomic features.

5. The method of claim 4 further comprising:
a) providing the genomic DNA sequence of a normal sample;
b) detecting the genomic features in the genomic DNA sequence of the normal sample;
c) comparing the genomic features in the genomic DNA sequence of the normal sample with those in the genomic DNA sequence of the cancer sample and
d) stratifying the patient into a cancer subgroup based on the increased prevalence of one or more of the genomic features in the genomic DNA sequence of the cancer sample compared to the genomic DNA sequence of the normal sample.

6. The method of claim 4 or 5 further comprising comparing the prevalence of one or more of the genomic features to a control.

7. A method for diagnosing a cancer in a subject in need thereof, the method comprising:
a. providing the genomic DNA sequence of a sample from the subject; and
b. detecting genomic features in the genomic DNA sequence of the sample, the genomic features including single nucleotide variants, insertions/deletions, mutation signatures, and structural variants,
wherein the prevalence of one or more of the genomic features is indicative of a diagnosis of a cancer.

8. The method of claim 7 further comprising:
a. providing the genomic DNA sequence of a normal sample;
b. detecting the genomic features in the genomic DNA sequence of the normal sample; and
c. comparing the genomic features in the genomic DNA sequence of the normal sample with those in the genomic DNA sequence of the sample from the subject
wherein the increased prevalence of one or more of the genomic features in the genomic DNA sequence of the sample from the subject compared to the genomic DNA sequence of the normal sample is indicative of a diagnosis of a cancer.

9. The method of claim 7 or 8 further comprising comparing the prevalence of one or more of the genomic features to a control.

10. The method of any one of claims 4 to 9 wherein the genomic features comprise a high level of fold-back inversions.

11. The method of claim 10 wherein the fold-back inversions co-localize with high-level amplifications.

12. The method of any one of claims 4 to 9 wherein the genomic features comprise a high level of insertions and deletions.

13. The method of any one of claims 4 to 12 further comprising determining a therapy for the cancer patient or the subject.

14. The method of any one of claims 4 to 13 wherein a high level of fold-back inversions stratifies the cancer patient or the subject into a subgroup susceptible to a therapeutic agent targeting a DNA repair mechanism.

15. The method of claim 14 wherein the subgroup susceptible to a therapeutic agent targeting a DNA repair mechanism is recalcitrant to therapy with cisplatin or a poly(ADP-ribose) polymerase inhibitor.

16. The method of claim 14 or 15 wherein the therapeutic agent is a DNA polymerase theta inhibitor.

17. The method of claim 16 wherein the therapy comprises sensitization to cisplatin or a poly(ADP-ribose) polymerase inhibitor.

18. The method of any one of claims 4 to 17 wherein the cancer patient or the subject has been previously exposed to genotoxic chemotherapy.

19. The method of any one of claims 1 to 18 wherein the cancer is a breast cancer or an ovarian cancer.

20. The method of claim 19 wherein the ovarian cancer is a high-grade serous carcinoma, is associated with endometriosis, or is a granulosa cell tumour.

21. The method of claim 20 wherein the ovarian cancer associated with endometriosis is an endometrioid carcinoma or a clear cell carcinoma.

22. The method of claim 21 wherein the ovarian cancer is a clear cell carcinoma subgroup susceptible to a therapeutic agent that targets an APOBEC enzyme.

23. The method of claim 21 wherein the ovarian cancer is an endometrioid carcinoma subgroup susceptible to immunotherapy.

24. The method of claim 19 wherein the breast cancer is a triple negative breast cancer.

25. The method of any one of claims 1 to 24 wherein the cancer is associated with a defect in a DNA repair mechanism.

26. The method of claim 25 wherein the DNA repair mechanism is a homologous recombination repair mechanism.

27. The method of claim 25 or 26 wherein the DNA repair mechanism is a microhomology-mediated end joining pathway.

28. The method of any one of claims 1 to 27 wherein the genomic DNA sequence is determined by whole genome sequencing.

29. The method of any one of claims 1 to 28 wherein the patient or the subject is a human.
